SEQUENCE LISTING (17 sequences)

SEQ ID NO: 1 - DNA, 7509 bp, Human immunodeficiency virus
tcgagagccccagttctacttacaccaggaaaggcttggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattaccgcgacagcagagatccagtttggaaaggaccagcaaagctcctctggaagggcgaaggggcagtagtaatacaagataatagtgacataaaagtagtgccaagaagaaaagcaaagatcatcagggattatggaaaacagatggcaggtgatgattgtgtggcaagtagacaggatgaggattaagggcccgtttaattaagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatcctagcgctcatacatatgccctagacaaaagccttaggcatctcctatggccggccgcagcggcgacagcgacgaggagctgatccgcaccgtgcgcctgatcaagctgctgtaccagagcgaccctccccctaaccctgagggcacccgccaggcccgccgcaaccgccggaggcggtggcgcgagcgccagcgccaaatccacagcatcagcgagcgcattctctcgacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttatctggatccaccggtgcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgagcggccgcccgggctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 2 - DNA, 4738 bp, Human immunodeficiency virus
tcgagagccccagttctacttacaccaggaaaggcttggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaatgaaggagagaacaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagatgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaaccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattaccgcgacagcagagatccagtttggaaaggaccagcaaagctcctctggaagggcgaaggggcagtagtaatacaagataatagtgacataaaagtagtgccaagaagaaaagcaaagatcatcagggattatggaaaacagatggcaggtgatgattgtgtggcaagtagacaggatgaggattaagggcccgtttaattaagatgttcatcaaatattactgggctgctattaacaagagatggtggtaataacaacaatgggtccgagatcttcagacctggaggaggcgatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaatcccgaggggacccgacaggcccgaaggaatagaagaagaaggtggagagagagacagagacagatccattcgattagtgaacggatcctagcgctcatacatatgccctagacaaaagccttaggcatctcctatggccggccgcagcggcgacagcgacgaggagctgatccgcaccgtgcgcctgatcaagctgctgtaccagagcgaccctccccctaaccctgagggcacccgccaggcccgccgcaaccgccggaggcggtggcgcgagcgccagcgccaaatccacagcatcagcgagcgcattctctcgacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttatctggatccaccggtgcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgagcggccgcccgggctcgagacctagaaaaacatggagcaatcacaagtagcaatacagcagctaacaatgctgcttgtgcctggctagaagcacaagaggaggaagaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 3 - DNA, 6304 bp, Human immunodeficiency virus
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggcgactggtgagtactatgggcgcccgcgccagcgtgctgagcggcggcgagctggacaaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattaccgcgacagcagagatccagtttggaaaggaccagcaaagctcctctggaagggcgaaggggcagtagtaatacaagataatagtgacataaaagtagtgccaagaagaaaagcaaagatcatcagggattatggaaaacagatggcaggtgatgattgtgtggcaagtagacaggatgaggattaagggcccgtgcaattggctttgcacctacaccagtaaaaagatactcctctgctccagtgaggaataaaagaggtgtattcgtgctagggttcttaggttttctcacgacagcaggagctgcaatgggcgcggcgtccttgacgctgtcggctcagtctcggactttattggccgggatagtgcagcaacagcaacagctgttggacgtggtcaagagacaacaagaaatgttgcgactgaccgtctggggaacaaaaaatctccaggcaagagtcactgctatcgagaaatacttaaaggaccaggcgcaactaaattcatggggatgtgcgtttagacaagtctgccacactactgtaccatgggtaaatgacaccttaacgcctgattggaacaacatgacatggcaggaatgggagcaacgaatccgcaacctagaggcaaatatcagtgaaagtttagaacaggcacaaatccagcaagaaaagaacatgtatgaactacaaaaattaaatagctgggatgtttttggcaactggtttgatttaacctcctggatcaaatatattcagtatggagtttatatagtagtaggaataatagttttaagaatagtaatatatgtagtacaaatgttaagtagacttagaaagggctataggcctgttttctcttccccccccgcttacttccaacagatccatacatatgccctagacaaaagccttaggcatctcctatggccggccgcagcggcgacagcgacgaggagctgatccgcaccgtgcgcctgatcaagctgctgtaccagagcgaccctccccctaaccctgagggcacccgccaggcccgccgcaaccgccggaggcggtggcgcgagcgccagcgccaaatccacagcatcagcgagcgcattctctcgacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttatctggatccttcgaacgtagctctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccattt

SEQ ID NO: 4 - DNA, 2540 bp, Human immunodeficiency virus
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggcgactggtgagtactatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagggcccgtgcaattggctttgcacctacaccagtaaaaagatactcctctgctccagtgaggaataaaagaggtgtattcgtgctagggttcttaggttttctcacgacagcaggagctgcaatgggcgcggcgtccttgacgctgtcggctcagtctcggactttattggccgggatagtgcagcaacagcaacagctgttggacgtggtcaagagacaacaagaaatgttgcgactgaccgtctggggaacaaaaaatctccaggcaagagtcactgctatcgagaaatacttaaaggaccaggcgcaactaaattcatggggatgtgcgtttagacaagtctgccacactactgtaccatgggtaaatgacaccttaacgcctgattggaacaacatgacatggcaggaatgggagcaacgaatccgcaacctagaggcaaatatcagtgaaagtttagaacaggcacaaatccagcaagaaaagaacatgtatgaactacaaaaattaaatagctgggatgtttttggcaactggtttgatttaacctcctggatcaaatatattcagtatggagtttatatagtagtaggaataatagttttaagaatagtaatatatgtagtacaaatgttaagtagacttagaaagggctataggcctgttttctcttccccccccgcttacttccaacagatccatacatatgccctagacaaaagccttaggcatctcctatggccggccgcagcggcgacagcgacgaggagctgatccgcaccgtgcgcctgatcaagctgctgtaccagagcgaccctccccctaaccctgagggcacccgccaggcccgccgcaaccgccggaggcggtggcgcgagcgccagcgccaaatccacagcatcagcgagcgcattctctcgacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttatctggatccttcgaacgtagctctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccattt

SEQ ID NO: 5 - DNA, 3477 bp, Human immunodeficiency virus
  misc_feature (3156)..(3156): n is a, c, g, or t
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtatcaattgtgagcgctcacaattgatacttagattcatcgagagggacacgtcgactactaaccttcttctctttcctacagctgagatcaccggcgaaggaggcctagatcaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattgatctatcgattgtacagctagattgagtcgacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggagccagtagatcctagactggagccctggaagcacccaggaagtcagcctaaaactgcttgtaccaattgctactgtaaaaagtgttgcttccactgtcaagtttgtttcatcaccaaggctttgggtatctcctacggtcgtaagaaacgtagacagcgcagacgtccaccgcaaggttctcagactcatcaagtttccttgtccaagcaaccgaccntcccaatctcgcggtgacccgacaggtcctaaggaatagtaaggatcgattgtacagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatcattt

SEQ ID NO: 6 - DNA, 10060 bp, Human immunodeficiency virus
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggcgactggtgagtactatgggcgcccgcgccagcgtgctgagcggcggcgagctggacaaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattaccgcgacagcagagatccagtttggaaaggaccagcaaagctcctctggaagggcgaaggggcagtagtaatacaagataatagtgacataaaagtagtgccaagaagaaaagcaaagatcatcagggattatggaaaacagatggcaggtgatgattgtgtggcaagtagacaggatgaggattaagggcccgtgcaattggctttgcacctacaccagtaaaaagatactcctctgctccagtgaggaataaaagaggtgtattcgtgctagggttcttaggttttctcacgacagcaggagctgcaatgggcgcggcgtccttgacgctgtcggctcagtctcggactttattggccgggatagtgcagcaacagcaacagctgttggacgtggtcaagagacaacaagaaatgttgcgactgaccgtctggggaacaaaaaatctccaggcaagagtcactgctatcgagaaatacttaaaggaccaggcgcaactaaattcatggggatgtgcgtttagacaagtctgccacactactgtaccatgggtaaatgacaccttaacgcctgattggaacaacatgacatggcaggaatgggagcaacgaatccgcaacctagaggcaaatatcagtgaaagtttagaacaggcacaaatccagcaagaaaagaacatgtatgaactacaaaaattaaatagctgggatgtttttggcaactggtttgatttaacctcctggatcaaatatattcagtatggagtttatatagtagtaggaataatagttttaagaatagtaatatatgtagtacaaatgttaagtagacttagaaagggctataggcctgttttctcttccccccccgcttacttccaacagatccatacatatgccctagacaaaagccttaggcatctcctatggccggccgcagcggcgacagcgacgaggagctgatccgcaccgtgcgcctgatcaagctgctgtaccagagcgaccctccccctaaccctgagggcacccgccaggcccgccgcaaccgccggaggcggtggcgcgagcgccagcgccaaatccacagcatcagcgagcgcattctctcgacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttatctggatccttcgaacgtagctctagattgagtcgacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggagccagtagatcctagactggagccctggaagcacccaggaagtcagcctaaaactgcttgtaccaattgctactgtaaaaagtgttgcttccactgtcaagtttgtttcatcaccaaggctttgggtatctcctacggtcgtaagaaacgtagacagcgcagacgtccaccgcaaggttctcagactcatcaagtttccttgtccaagcaaccgacctcccaatctcgcggtgacccgacaggtcctaaggaatagtaaggatcgatccgaattcgctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgcgcagggcccaattgctcgagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatgcgcgctcgcgaaccaggttcctgtcaccggatgtgctttccggtctgatgagtccgtgaggacgaaacaggactcccggggcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgtaagtgatatctactagatttatcaaaaagagtatcaattgtgagcgctcacaattgatacttagattcatcgagagggacacgtcgactactaaccttcttctctttcctacagctgagatcaccggcgaaggaggcctagatcaattctgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatgaaaaaaaaaaaaaaaaacggaattgatctatcgattgtacagctagctcgacatgataagatacattgatgagtttggacaaaccacaactagaatgcagtgaaaaaaatgctttatttgtgaaatttgtgatgctattgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagatccattt

SEQ ID NO: 7 - DNA, 116 bp, Human immunodeficiency virus
ctcggcttgctgaagcgcgcacggcaagaggcgaggggcggcgactggtgagtacgccaaaaattttgactagcggaggctagaaggagagagatgggtgcgagagcgtcggtatt

SEQ ID NO: 8 - DNA, 634 bp, Human immunodeficiency virus
tggaagggctaatttggtcccaaaaaagacaagagatccttgatctgtggatctaccacacacaaggctacttccctgattggcagaactacacaccagggccagggatcagatatccactgacctttggatggtgcttcaagttagtaccagttgaaccagagcaagtagaagaggccaaataaggagagaagaacagcttgttacaccctatgagccagcatgggatggaggacccggagggagaagtattagtgtggaagtttgacagcctcctagcatttcgtcacatggcccgagagctgcatccggagtactacaaagactgctgacatcgagctttctacaagggactttccgctggggactttccagggaggtgtggcctgggcgggactggggagtggcgagccctcagatgctacatataagcagctgctttttgcctgtactgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagca

SEQ ID NO: 9 - DNA, 634 bp, Human immunodeficiency virus
tggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatggaatggatgaccctgagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatgctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 10 - DNA, 348 bp, Human immunodeficiency virus
atggccggccgcagcggcgacagcgacgaggagctgatccgcaccgtgcgcctgatcaagctgctgtaccagagcgaccctccccctaaccctgagggcacccgccaggcccgccgcaaccgccggaggcggtggcgcgagcgccagcgccaaatccacagcatcagcgagcgcattctctcgacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaa

SEQ ID NO: 11 - DNA, 245 bp, Human immunodeficiency virus
gcagtgggaataggagctttgttccttgggttcttgggagcagcaggaagcactatgggcgcagcgtcaatgacgctgacggtacaggccagacaattattgtctgatatagtgcagcagcagaacaatttgctgagggctattgaggcgcaacagcatctgttgcaactcacagtctggggcatcaaacagctccaggcaagaatcctggctgtggaaagatacctaaaggatcaacagctcct

SEQ ID NO: 12 - DNA, 118 bp, Human immunodeficiency virus
ctcggcttgctgaagcgcgcacggcaagaggcgaggggcggcgactggtgagtacgccaaaaattttgactagcggaggctagaaggagagagatgggtgcgagagcgtcggtattaa

SEQ ID NO: 13 - DNA, 190 bp, Human immunodeficiency virus
agacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaa

SEQ ID NO: 14 - DNA, 15 bp, Human immunodeficiency virus
aaaagaaaagggggg

SEQ ID NO: 15 - DNA, 4307 bp, Human immunodeficiency virus
atgggtgcgagagcgtcggtattaagcgggggagaattagataaatgggaaaaaattcggttaaggccagggggaaagaaacaatataaactaaaacatatagtatgggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaatgcatgggtaaaagtagtagaagagaaggctttcagcccagaagtaatacccatgttttcagcattatcagaaggagccaccccacaagatttaaataccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataaagataggggggcaattaaaggaagctctattagatacaggagcagatgatacagtattagaagaaatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 16 - DNA, 16 bp, Human immunodeficiency virus
taatttttttagggaa

SEQ ID NO: 17 - DNA, 6 bp, Human immunodeficiency virus
atgggt

(19) United States
(12) Patent Application Publication
(10) Pub. No.: US 2011/0142880 A1
(43) Pub. Date: Jun. 16, 2011

(54) LENTIVIRUS-BASED IMMUNOGENIC VECTORS

(76) Inventors: Franck Yann Lemiale, North Potomac, MD (US); Nikolay Korokhov, Germantown, MD (US); Laurent M. Humeau, Germantown, MD (US); Vladimir Slepushkin, Damascus, MD (US)

(21) Appl. No.: 12/934,987

(22) PCT Filed: Mar. 27, 2009
(86) PCT No.: PCT/US09/38535

§ 371 Date: Feb. 28, 2011

Related U.S. Application Data
(60) Provisional application No. 61/040,581, filed on Mar. 28, 2008.

(51) Int. Cl. A61K 39/21; C12N 15/63; C12N 15/867; A61K 31/7088; C12N 5/10; A61P 37/04; A61P 35/00; A61P 25/28; A61P 9/00; A61P 25/00; A61P 3/00; A61P 19/02

(52) U.S. Cl. 424/207.1; 435/320.1; 435/455; 514/44 R; 435/325

(57) ABSTRACT

The present invention provides for lentiviral vectors for vaccine delivery comprising a 5′ long terminal repeat (LTR) and a 3′ LTR, an integrated nucleic acid sequence, wherein the integrated nucleic acid sequence is expressed by the 5′ LTR; and a nucleic acid sequence encoding functional REV coding sequence and a rev response element (RRE)-containing sequence, wherein the RRE-containing sequence is located upstream of the REV coding sequence, and wherein transcription of said first nucleic acid sequence and said second nucleic acid sequence is driven by said 5′ LTR. Also provided for are pharmaceutical compositions, methods of making and using the lentiviral vectors of the present invention.

### CROSS REFERENCE TO RELATED APPLICATIONS

This application claims priority to provisional application No. 61/040,581 filed Mar. 28, 2008, the entire contents of which are incorporated herein in their entirety.

### FIELD OF THE INVENTION

This invention describes improved lentiviral-based immunogenic vectors for use in prophylactic and therapeutic treatment of viral infection. This invention more particularly relates to a lentiviral vector, methods of making, modifying, propagating, packaging the same, and methods of using such a lentiviral vector as a vaccine for human immunodeficiency virus (HIV) infection and other diseases.

### BACKGROUND OF THE INVENTION

Nearly three decades following the first identification of acquired immunodeficiency syndrome (AIDS) in humans and the human immunodeficiency virus (HIV) causing the disease, more than 33 million people are living with HIV worldwide, and over four million people become newly infected with HIV yearly, while an estimated 2.8 million have already lost their lives to AIDS. As the HIV pandemic continues to spread world-wide, the need for an effective HIV vaccine remains urgent. The extraordinary ability of HIV to mutate, the inability of many currently known specificities of anti-HIV antibodies to consistently neutralize HIV primary isolates, and the lack of a complete understanding of the correlates of protective immunity to HIV infection have impeded efforts to develop an HIV vaccine having the desired effectiveness. At present, no vaccine has been shown to be truly effective against HIV, just as sterilizing immunity to the virus remains virtually impossible. Therefore, a substantial un-met need exists for an effective vaccine not only as a palliative treatment for AIDS, but also for reducing and possibly abolishing the transmission of the virus. Although a majority of HIV-infected subjects develop acquired immunodeficiency syndrome (AIDS), approximately 10-15% of patients are AIDS-free after 10 years of infection, and are termed non-progressors to AIDS (Sheppard et al., AIDS 7:1159 66 (1993), Phair, AIDS Res. Human Retroviruses 10:883 885 (1994)). Of those that do develop AIDS, approximately 10% of HIV-infected patients progress to AIDS within the first two to three years of HIV infection, and are termed rapid progressors to AIDS (Sheppard et al., AIDS 7:1159 66 (1993), Phair, AIDS Res. Human Retroviruses 10:883 885 (1994)). The initial characterization of anti-HIV immune responses in non-progressors and rapid progressors to AIDS has provided some insight into what may be the correlates of protective immunity to HIV.

In general, rapid progressors to AIDS have lower levels of antibodies to HIV proteins (Sheppard et al., AIDS 7:1159 66 (1993), Pantaleo et al., N. Engl. J. Med. 332:209 216 (1995), Cao et al., N. Eng. J. Med. 332:201 208 (1995)), and low or absent antibodies that neutralize autologous HIV isolates (Pantaleo et al., N. Engl. J. Med. 332:209 216 (1995), Cao et al., N. Eng. J. Med. 332:201 208 (1995)). Plasma levels of HIV virions are generally higher in rapid progressors compared to non-progressors, and rapidly replicating HIV strains are isolated more frequently from rapid progressors (Lee et al., J. AIDS 7:381 388 (1994), Mellors et al., Ann. Intern. Med. 122:573 579 (1995), Jurriaans et al., Virology 204:223 233 (1994)), either as a consequence of immunodeficiency and selection of more virulent HIV variants, or as a consequence of more virulent HIV variants infecting rapid progressors (Sullivan et al., J. Virol. 69:4413 4422 (1995)). Taken together with data that the fall in plasma viremia in primary HIV infection correlates with the presence of CD8+ anti-HIV cytotoxic T-cell lymphocyte (“CTL”) activity (Borrow et al., J. Virol. 68:6103 (1994)), these data suggest that anti-HIV CD8+ CTL that kill HIV-infected cells and antibodies that broadly neutralize HIV primary isolates may provide therapeutic anti-HIV immune responses in uninfected individuals subsequently exposed to HIV (Haynes et al., Science 271:324 328 (1996), Haynes, Science 260:1279 1286 (1993)).

Historically, vaccines provide protection from virus infection by eliciting a strong antiviral neutralizing antibody response. Neutralizing antibodies recognize proteins on the virus surface and prevent binding to and infection of healthy cells. However, this approach is not effective against HIV due to the broad range of HIV subtypes and rapid mutation rate that allows HIV to escape immune responses that are not sufficiently diverse. Some of the most successful of vaccines designed to elicit neutralizing antibody consist of recombinant envelope proteins derived from HIV. Poor protection by vaccines may be a result of an inability thus far to stimulate a robust and diverse cellular and humoral immune response. Researchers are also developing HIV vaccines based upon the cellular immune response. Cellular immunity is based upon the CTLs, or CD8+ T lymphocytes, which kill cells that are infected with virus. This approach prevents amplification of virus in the body so that disease does not develop and the virus is less likely to be transmitted to another individual.

First generation HIV vaccines currently in development may offer some form of protection, but they will not be entirely protective. Prophylaxis to disease is unlikely to occur with first-generation prototypes of AIDS vaccines. More likely, these vaccines will affect the clinical course of the disease, not prevent infection, and reduce the viral load and prolong symptom alleviation or symptom-free survival by slowing progression to AIDS. Finally, such a vaccine may affect person to person transmission, since high viral loads have been strongly correlated to increased rates of HIV transmission. See, e.g., Holodniy 2006.

Therefore, a vaccine may reduce viral load to a level that results in decreased transmission. A vaccine on an individual level may therefore be extremely beneficial on an epidemiological level, at a scale of a whole population. However, even a partially effective vaccine could be very promising in protecting some individuals against infection. In addition to reducing the rate of transmission of HIV, lowering viral load in infected individuals, would slow progression to AIDS and prolong life expectancy.

Two biological features of the virus have posed the biggest impediments to developing a vaccine: its extraordinary genetic diversity and the evasive properties of its envelope proteins. See, e.g., Brander, Frahm et al. 2006; Butler, Pandrea et al. 2007; and McBurney and Ross 2007. Previous vaccine initiatives, focusing on mounting an immune response to the HIV envelope protein, have largely failed due to these features of HIV. A recent example that failed was a vaccine containing a synthetic gp120 with little effectiveness overall. Another vaccine trial using a trivalent adenovirus vaccine was abruptly suspended for failure to offer prophylactic or therapeutic effect in vaccine recipients as well.

Accordingly, a continuing and unmet need exists for new, improved, and alternative prophylactic and therapeutic treatment modalities for HIV infection.

Citation of the above documents or any references cited herein is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present invention provides lentiviral vectors, methods of making, modifying, propagating, and packaging such lentiviral vectors, and methods of using such vectors to potentiate an immune response against one or more diseases in a subject.

In one aspect, the present invention provides for improved lentiviral vectors for vaccine delivery comprising a 5′ long terminal repeat (LTR) and a 3′ LTR; a first nucleic acid sequence operably linked to said 5′ LTR, also referred to herein as the “payload”; and a second nucleic acid sequence, that is operably linked to said 5′ LTR comprising a functional REV coding sequence and a rev response element (RRE)-containing sequence, wherein the RRE-containing sequence is located upstream of the REV coding sequence, and wherein transcription of said first nucleic acid sequence and said second nucleic acid sequence is driven by said 5′ LTR.

In one embodiment, the present invention provides for improved lentiviral vectors for vaccine delivery comprising a 5′ long terminal repeat (LTR) (SEQ ID NO: 8) and a 3′ LTR (SEQ ID NO: 9); a first nucleic acid sequence operably linked to said 5′ LTR, also referred to herein as the “payload”; and a second nucleic acid sequence, that is operably linked to said 5′ LTR comprising a functional REV coding sequence (SEQ ID NO: 10) and a rev response element (RRE) (SEQ ID NO: 11)-containing sequence wherein the RRE-containing sequence is located upstream of the REV coding sequence, and wherein transcription of said first nucleic acid sequence and said second nucleic acid sequence is driven by said 5′ LTR. The present invention is characterized in that expression of the payload is driven by the 5′LTR and also where expression of the payload depends on the activity of Rev and the RRE-containing sequence. In other words, the 5′ LTR can be a powerful enough promoter to drive expression of the payload and REV.

In yet another embodiment, such lentiviral vectors may comprise a 5′ LTR from a lentivirus; a 3′ LTR from a lentivirus; a first nucleic acid sequence, i.e., a payload; and a second nucleic acid sequence, that is operably linked to said 5′ LTR an RRE-containing sequence and a REV coding sequence wherein the RRE-containing sequence is located upstream of the REV coding sequence, wherein the first nucleic acid sequence expresses the full length wild-type sequence of either one or both of Gag and Pol; and wherein transcription of said first nucleic acid sequence and said second nucleic acid sequence is driven by said 5′ LTR.

In yet another embodiment, the lentiviral vectors for vaccine delivery comprise a 5′ long terminal repeat (LTR) and a 3′ LTR; a first nucleic acid sequence operably linked to said 5′ LTR; and a second nucleic acid sequence that is operably linked to said 5′ LTR comprising a functional REV coding sequence and a rev response element (RRE)-containing sequence, wherein the RRE-containing sequence is located upstream of the REV coding sequence, and wherein transcription of said first nucleic acid sequence and said second nucleic acid sequence is driven by said 5′ LTR, and wherein the lentiviral vector construct may further comprise one or more of the known lentiviral regulatory elements such as Tat, Nef, Vif, Vpu, Vpr or a combination thereof.

In yet another embodiment of the present invention, the lentiviral vectors for vaccine delivery specifically exclude all known lentiviral regulatory elements such as Tat, Nef, Vif, Vpu, and Vpr. In such an embodiment, the nucleic acid sequence(s) encoding at least one of Vpu, Vpr, Vif, Tat, Nef, or analogous lentiviral proteins are disrupted such that the nucleic acid sequence(s) are incapable of encoding functional Vpu, Vpr, Vif, Tat, Nef, or the nucleic acid sequence(s) encoding Vpu, Vpr, Vif, Tat, Nef, or analogous auxiliary proteins are removed from the lentiviral vector system of the present invention.

In another aspect, the invention provides for a pharmaceutical composition for in vitro or in vivo administration comprising a lentiviral vector of the present invention; and a pharmaceutically acceptable carrier and/or a genetic adjuvant as are known in the art.

In another aspect, the invention provides for a method of increasing the immunogenicity of a vector in a host cell comprising administering a pharmaceutical composition comprising a first vector; and sequentially administering a pharmaceutical composition comprising a second lentiviral vector comprising a 5′ long terminal repeat (LTR) and a 3′ LTR; a first nucleic acid sequence operably linked to said 5′ LTR; and a second nucleic acid sequence operably linked to said 5′ LTR comprising a functional REV coding sequence and rev response element (RRE)-containing sequence wherein the RRE-containing sequence is located upstream of the REV coding sequence, wherein transcription of said first nucleic acid sequence and said second nucleic acid sequence is driven by said 5′ LTR, and wherein the immunogenicity of said vector is increased.

In yet another aspect, the invention provides for a method for inducing an immune response in a subject, said method comprising administering a pharmaceutical composition comprising the lentiviral vectors disclosed herein, by various administration routes and protocols, e.g., DNA prime/lentiviral vector boost, DNA prime/two homologous lentiviral vector boosts, DNA prime/heterologous vector boost, lentiviral prime/lentiviral vector boost, lentiviral vector or DNA prime/lentiviral vector boost/adenoviral vector boost and vice versa, where the adenoviral vector is administered as the first boost and the lentiviral vector is administered as the second boost.

Thus in one embodiment, the method provides for the generation of both a cell mediated and humoral immune response against HIV, or other disease of interest relating to antigenic or immunogenic sequences encoded in the construct, in a subject by administration of the lentiviral vaccine vectors disclosed herein by various administration routes and protocols.

In yet another embodiment of the invention, a method for inducing an immune response in a subject comprising administration of the lentiviral vaccine vector of the present invention is provided, wherein diversified immunity is elicited. This includes cellular or humoral immunity either systemically or mucosally exhibited.

It is yet another embodiment of the invention to provide for a prime/boost or heterologous or homologous boost approach to increase the effectiveness and/or immunogenicity of a vector comprising administering a first vector (e.g., a nucleic acid plasmid construct, referred to as a DNA prime plasmid or using lentiviral vectors of the present invention, adenoviral-based vectors, pox-based vectors, including MVA and canary pox, VSV-based vectors, alphavirus-based vectors (e.g., VEE, Semliki Forest Virus), herpes virus-based vectors, among others known in the art) and subsequently administering a lentiviral vector of the present invention as a boost comprising the same payload, wherein one or both first and second vectors are the lentiviral vector of the present invention as a boost comprising the same payload as the DNA prime plasmid construct, or in some cases, a lentiviral vector of the present invention as the prime of a prime/boost protocol followed by a viral vector boost (for example, employing adenoviral-based vectors, pox-based vectors, including MVA and canary pox, VSV-based vectors, alphavirus-based vectors (e.g., VEE, Semliki Forest Virus), herpes virus-based vectors), or vice versa.

In yet another aspect of the invention, a method is provided for inhibiting the replication of an infective replicative human immunodeficiency virus (HIV) in a host comprising: administering a pharmaceutical composition comprising: a 5′ long terminal repeat (LTR) and a 3′ LTR; a first nucleic acid sequence operably linked to said 5′ LTR; and a second nucleic acid sequence operably linked to said 5′ LTR comprising a functional REV coding sequence and rev response element (RRE)-containing sequence wherein the RRE-containing sequence is located upstream of the REV coding sequence, and wherein transcription of said first nucleic acid sequence and said second nucleic acid sequence is driven by said 5′ LTR.

In yet another aspect of the present invention, a method is provided for prophylactically or therapeutically treating a subject for a viral, bacterial, parasitic and/or microbial infection comprising administering to a subject in need thereof of a lentiviral vectors comprising a 5′ long terminal repeat (LTR) and a 3′ LTR; a first nucleic acid sequence operably linked to said 5′ LTR; and a second nucleic acid sequence, that is operably linked to said 5′ LTR comprising a functional REV coding sequence and a rev response element (RRE)-containing sequence, wherein the RRE-containing sequence is located upstream of the REV coding sequence, and wherein transcription of said first nucleic acid sequence and said second nucleic acid sequence is driven by said 5′ LTR.

Such methods can additionally comprise the use of a prime/lentiviral vector boost approach, as well as the other protocols disclosed herein. The method can be used, for example, to generate a diverse and comprehensive immune response in a subject against a disease of interest, including cancer, or other disease conditions such as for example, and not by way of limitation, Alzheimer's disease, autoimmune diseases, cardiovascular diseases, neurological diseases, fibrotic diseases, lipid metabolism diseases, extra-cellular matrix-related diseases, and chronic joint degenerative diseases, or a combination thereof.

In yet another aspect of the present invention, the invention provides for a packaging cell line and method of making a packaging cell line for making the vaccine vectors of the present invention. In one embodiment, a method of producing a recombinant lentiviral packaging cell is provided comprising introducing into a cell, a nucleic acid capable of expressing in said packaging cell, a nucleic acid sequence to produce transduction-competent virus-like particles; and at least one nucleic acid molecule capable of expressing the sequence of interest in said packaging cell, wherein said packaging cell produces transduction-competent virus-like particles expressing the nucleic acid sequence of interest.

In each of the aforementioned lentiviral vectors, pharmaceutical compositions containing such lentiviral vectors, and methods of using such lentiviral vectors, the lentiviral vector further comprises one or more of the following including, for example, and not by way of limitation, a nucleic acid sequence encoding functionally active lentiviral RNA packaging elements, a nucleic acid sequence encoding functional central polypurine tract (cPPT), a central termination sequence (CTS) and 3′ LTR proximal polypurine tract (PPT), and/or a nucleic acid sequence encoding a non-protein or protein based marker or tag. In specific embodiments, the lentiviral vector of the present invention comprises one or more of the lentiviral vector constructs depicted in FIG. 1, FIG. 2 or FIG. 3, or any combination thereof.

Additionally, the lentiviral vectors of the present invention may include one or more of the following characteristics. Thus, in one embodiment of the present invention, the first nucleic acid sequence encodes one or more antigenic sequences of interest. In yet another embodiment of the present invention, the expression of one or more sequences of interest depends on REV-RRE activity. In yet another embodiment of the present invention, the first nucleic acid sequence comprises a gag/pol coding sequence. In yet another embodiment of the present invention, the first nucleic sequence or second nucleic acid sequence is an unmodified sequence. In yet another embodiment of the present invention, the gag coding sequence comprises a modified gag coding sequence. In yet another embodiment of the present invention, the modified gag coding sequence further comprises at least one nucleotide substitution which alters the myristylation receptor glycine residue of Gag. In yet another embodiment of the present invention, the gag/pol encoding sequence is modified to increase cellular expression. In yet another embodiment of the present invention, the modified sequence comprises a frameshift within the gag sequence. In yet another embodiment of the present invention, the modification supports a high level of expression of gag-pol fusion protein not mediated by translational frameshifting. In yet another embodiment of the present invention, the cPPT is a part of a pol coding sequence of the gag/pol coding sequence. In some embodiments of the present invention, the functionally active lentiviral RNA packaging elements comprise a gag packaging sequence.

These and other aspects of some exemplary embodiments will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments without departing from the spirit thereof.

### BRIEF DESCRIPTION OF THE DRAWINGS

FIG. 1 depicts an illustrative HIV vaccine candidate and various analogs thereof. This candidate is an HIV-1 based lentiviral vector (LV) using the native HIV LTR as promoter. It expresses gag, pol and rev genes as immunogenic payloads. An important feature of this LV resides in the engineering or placement of the RRE-containing sequence upstream of the full length Rev coding sequence. Another important feature of this LV is the presence of a splice acceptor (SA) site located between the RRE-containing sequence and the Rev coding sequence. Another important feature of this LV is the immediate proximity of the immunogenic payload(s) to the RRE-containing sequence. This LV optionally contains a non-protein coding genetic sequence (Gtag) to allow its identification and discrimination from wt-HIV in an HIV therapeutic vaccine setting. This sequence can be placed either upstream of downstream of the SA site. The four other schematic representations depicted in FIG. 1 are non-limiting representative examples of possible LV vaccine vector analogs. The second construct shows that the RRE element can be placed upstream or downstream of the immunogenic payload(s). The third construct shows that the Gtag sequence can be disposable. The fourth construct shows that downstream of the sequence encoding for the Rev protein another genetic payload(s) can be placed, payloads able to encode for genetic adjuvant, other immunogens, RNA antisense, ribozymes, etc. The fifth construct is a combination of the schematic vector construct representations described supra. The final and sixth construct contains an additional element which can be introduced for an additional regulation of transgene expression where SD/SA sites will be optional. An internal promoter/Rev combination could be used in all above listed constructs.

FIG. 2 depicts a generic vaccine HIV-based lentiviral vector construct and analogs thereof. The generic vaccine vector is an HIV-1 based lentiviral vector (LV) using the native HIV LTR as promoter. It contains the minimal elements for production, encapsidation and integration in transduced host cells (psi, cPPT/cts and ppt elements). As for the HIV vaccine vector, the important feature of this LV resides in the engineering or placement of the RRE-containing sequence upstream of the full length Rev coding sequence, the placement of the splice acceptor (SA) site between the RRE-containing sequence and the Rev coding sequence, and the immediate proximity of the immunogenic payload(s) to the RRE sequence. This LV contains a non-protein coding genetic sequence (Gtag) to allow its identification and discrimination from wt-HIV in an HIV therapeutic vaccine setting. This sequence can be placed either upstream of downstream of the SA site. The three other schematic representations are several non-limiting examples of possible LV generic vector analogs. The second construct shows that the RRE element can be placed upstream or downstream of the genetic payload(s). The third construct shows that the Gtag sequence can be disposable. The fourth construct shows that downstream of the sequence encoding for the Rev protein another genetic payload(s) can be placed, payloads able to encode for genetic adjuvant, other immunogens, RNA antisense, ribozymes, etc. The final and fifth construct shows a configuration with an internal promoter regulated Rev and adjoined gene expression. As for the last example in FIG. 1A, the presence of SD/SA sites could be optional.

FIG. 3 depicts HIV vaccine candidate and analogs using a SIN configuration. This figure present schematic representation of the HIV vaccine vector candidate (first from top) described in the application. This candidate is an HIV-1 based lentiviral vector (LV) with the native HIV LTR promoter activity disrupted either by deletion, mutation or insertion of elements such as Insulators. This SIN based HIV vaccine LV expresses gag, pol and rev genes as immunogenic payloads. As for the HIV vaccine vector, the important feature of this LV resides in the engineering or placement of the RRE-containing sequence upstream of the full length Rev coding sequence, the placement of the splice acceptor (SA) site between the RRE-containing sequence and the Rev coding sequence, and the immediate proximity of the immunogenic payload(s) to the RRE sequence. This LV contains a non-protein coding genetic sequence (Gtag) to allow its identification and discrimination from wt-HIV in HIV therapeutic vaccine setting. This sequence can be placed either upstream of downstream of the SA site. The four other schematic representations are non-limiting examples of possible SIN-based LV vaccine vector analogs. The second construct shows that the RRE element can be placed upstream or downstream of the immunogenic payload(s). The third construct shows that the Gtag sequence can be disposable. The fourth construct from the top shows that downstream of the sequence encoding for the Rev protein another genetic payload(s) can be placed, payloads able to encode for genetic adjuvant, other immunogens, RNA antisense, ribozymes, etc. The final and fifth construct is a combination of the schematic vector construct representations described supra.

FIGS. 4A, 4B, 4C and 4D depict the results of a sub-cutaneous (SC) HIV vaccine lentivirus candidate (for example, referred to herein as “VRX1023”) immunization of mice inducing both systemic and mucosal humoral and cellular immunity. Mice were immunized twice at days 0 and 15 with 2×10⁷ TU of VRX1023 injected SC, then sacrificed at day 25 (Panel A). The negative control group received 2×10⁷ TU of a lentiviral vector carrying EGFP in place of the Gag, Pol and Rev transgenes. Anti-HIV antibodies were detected at the systemic (serum, Panel B) and mucosal level (saliva, panel C) by Elisa. CD8+ T-cells expressing cytokines (IFNγ, TNFα, IL2 combined on the same channel) after HIV-Gag peptides stimulation were detected by ICS (panel D). Results are shown as individual values (dots) and geometric mean in each group (bars).

FIG. 5 depicts that VRX1023 immunogenicity can be significantly improved in DNA prime, vector boost settings. Groups of 5 mice were immunized with either PBS, VRX1053 DNA (another HIV vaccine lentivirus candidate), VRX1023 LV, or a combination of these. When immunized with DNA, mice received 100 ug of pVRX1053 IM. For LV immunization, mice received 10⁹ TU of VRX1023 SC. Anti-HIV CD8+ T-cell responses were assessed by ICS after stimulation of splenocytes with Gag and Pol peptide pools. Bars indicate, for each animal, the percentage of cells staining positively both for cytokines (IFNγ, TNFα, IL2 combined on the same channel) and for the activation marker CD69, within the CD8+ subset of CD3+ lymphocytes.

FIG. 6 illustrates that VRX1023 immunogenicity can be significantly improved by vector manufacturing optimizations. Groups of 5 mice were immunized with pVRX1053 DNA prime (100 ug of pVRX1053 IM, three times), followed by VRX1023 LV boost (10⁹ TU of VRX1023 SC, 2 weeks after prime). Figure shows examples of cytokine secretions as an illustration of immunogenicity improvement provided by LV manufacturing optimization. Each set of Gag-induced cytokine secretion is the result of independent experiments. The lower right panel illustrates that by using an optimized lentiviral vector, a robust cellular immunity was elicited with up to 21.11% of murine CD8+ cells recognizing the HIV-Gag antigen and responding by secreting cytokines (IFNγ, TNFα, IL2), while the vaccine dose used was lower.

FIG. 7 depicts the long-term cell mediated and humoral anti-HIV immunity elicited by the vectors and prime/boost protocols of the invention. Three groups of five mice were immunized twice at days 0 and 15 with 8×10⁷ Transducing Units (TU) of VRX1023 injected sub-cutaneously (SC). Each group was sacrificed at 10 days, 3 weeks and 2 months post-last immunization, respectively (Panel A). T-cell responses to HIV-Gag (dot plots and left y-axis) were measured by Intracellular Cytokine Staining (ICS). Splenocytes were stimulated in vitro with Gag peptides. Panel B shows the percentage of cytokine positive cells (IFNγ, TNFα, IL2 combined on the same channel) out of the CD3+ CD8+ T cell subset. The percentage of cytokine producing cells is shown for each individual animal (dots). Bars represent geometric means. Serum samples collected at time of sacrifice in each group was analyzed by Elisa for anti-HIV IgG detection (red line graph and right y-axis). Data are shown, for each time point, as geometric means, along with standard deviations.

FIG. 8 depicts a comparison data between prime/boost protocols where the prime was a DNA plasmid containing the same HIV payload as either a lentiviral vaccine vector or adenoviral vectors. At equal doses, LV induces higher anti-HIV immunity than human adenovirus serotype 5 (Ad5). Groups of five mice were immunized with three DNA primes (100 μg DNA IM/animal), followed by a vector boost (10⁸ TU/animal, SC) according to the schedule described on panel A. Head to head comparisons were performed between LV and Ad5 candidates from two sources. In panel B, mice were primed with a plasmid encoding the multi HIV payload, then boosted with equal doses of either the LV VRX-multi HIV or with Ad5-multi HIV. Animals were sacrificed 1 month post-immunization and samples collected for analysis. T-cell responses (bars, left y-axis) are measured by ICS to HIV-Gag and are shown as geometric means, along with standard deviations (percentages of cytokine positive cells (IFNγ, TNFα, IL2 combined on the same channel) within the CD8+ lymphocyte subset). Anti-HIV IgG were detected in the sera at 1:100 by Elisa (line graph; right y-axis). Results shown represent the ratio of the antibody response at time of sacrifice to the pre-immunization baselines'. In panels C and D, a second Ad5 candidate, VRC-Ad5, containing the same Gag-Pol transgene as its lentiviral counterpart, VRX1023, was evaluated. All animals were primed with the same pVRX1053 plasmid before boosting with equal doses of either vector. Panel C illustrates anti-HIV cellular responses obtained at 2 months post-vector boost with each candidate, and represents the cumulative geometric means and standard deviations within each group. Values are percentages of cells secreting all three cytokines simultaneously among CD4 T cells (polyfunctional T cells). Anti-HIV IgG responses in sera were assessed by Elisa at one and two months post-vector boost (Panel D). Lines represent geometric means in each group, along with standard deviations. Pre-immunization baseline values, similar among all animals, were averaged and showed as a dotted line.

FIG. 9 depicts comparison data of anti-HIV cell mediated responses of CD8+ cells that were isolated from mice vaccinated with five different vaccine protocols. Heterologous immunizations elicit the greatest cumulative HIV peptide-specific cytokine responses in CD8+ T cells. Groups of 5 mice were subjected to homologous immunizations with VRX1023 or Ad5, or heterologous immunizations using combinations of both vectors. Following stimulation with HIV Gag (top panels) or Pol (bottom panels) peptide mixes, production of IFNγ (left panels) or TNFα (right panels) was quantified by enumerating cytokine positive cells gated on CD3+ CD8+ T cells. Each point represents individual samples, and the bars correspond to the average of each sample group.

FIG. 10 depicts T cell polyfunctionality in response to HIV-Pol stimulation in CD8 T-cells, as measured by ICS. Samples were gated for T cells which secreted IFNγ, TNFα as well as IL2, an indication of polyfunctionality.

FIG. 11 depicts that anti-vector antibodies neutralize lentiviral vectors very poorly compared to adenoviral vectors; heterologous prime/boost protocols bypass most of the anti-Ad5 neutralization. Neutralizing activity in immunized mice against VRX1023 or VRC-Ad5 was assayed by incubating a lentiviral HIV vector expressing GFP (for example, VRX494-GFP) and Ad5-GFP, respectively, at MOI 10 with mouse sera acquired 17 days post-immunization, and diluted 80-fold. Vector neutralization was calculated as a function of down-regulation of GFP expression in post-immunization sera relative to GFP expression in pre-immunization sera. Each point represents individual samples, and the bars correspond to the average of each sample group.

FIG. 12 illustrates the immunogenicity of various DNA prime, heterologous boost protocols. Mice were vaccinated with two different DNA prime/heterologous boost protocols. CD8+ immune response to two HIV antigens, Gag and Pol, were measured as the percentage of CD8+ cells that secrete either of the cytokines tested (INFγ and TNFα).

FIG. 13 illustrates the level of anti-SIV p27 antibody titers generated in non-human primates after an SIV-(primate equivalent of HIV) lentiviral-based vaccine vector was administered three times over five months. The boosting effect observed after the third administration reveals a minimal anti-vector response even after multiple administrations.

FIG. 14A depicts a packaging helper vector construct for the lentiviral vaccine vectors of the present invention where the lentiviral vaccine vector of the invention contains a Gag-pol payload. FIG. 14B depicts a packaging helper vector construct for lentiviral vaccine vectors where the payload of the vaccine vector is different from full length Gag-pol. This helper construct supplied everything needed to package the lentiviral vaccine vector.

FIGS. 15 and 16 depict packaging cell line helper constructs for vaccine lentiviral vector production where the payload nucleotide is gag/pol and where the payload is a nucleotide sequence is other than gag/pol.

### BRIEF DESCRIPTION OF THE SEQUENCE LISTING

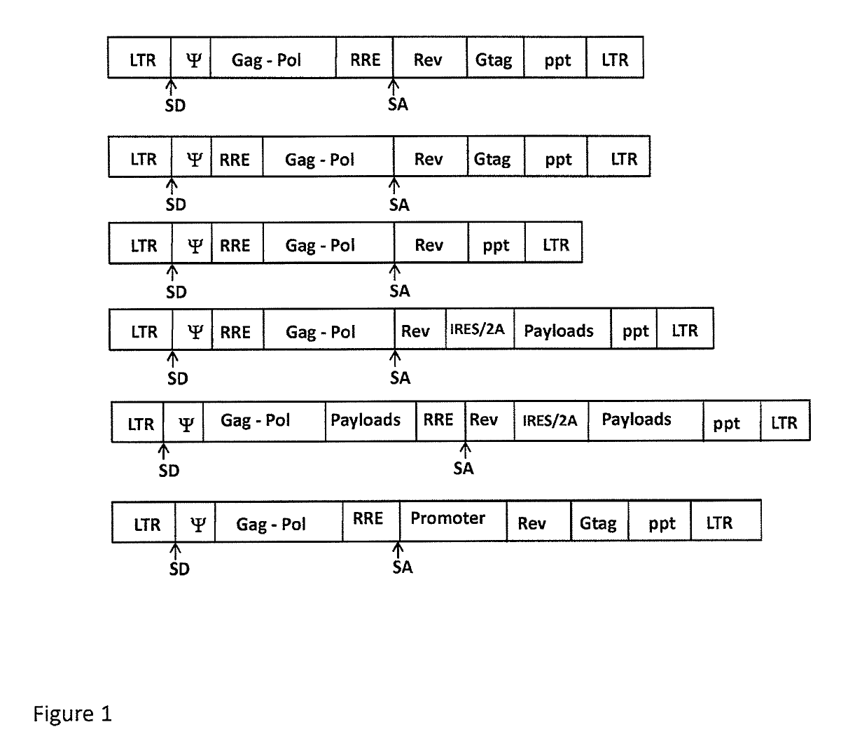

SEQ ID NO: 1 is an example of an HIV vaccine vector of FIG. 1A.

SEQ ID NO: 2 is an example of a vaccine vector of FIG. 1B.

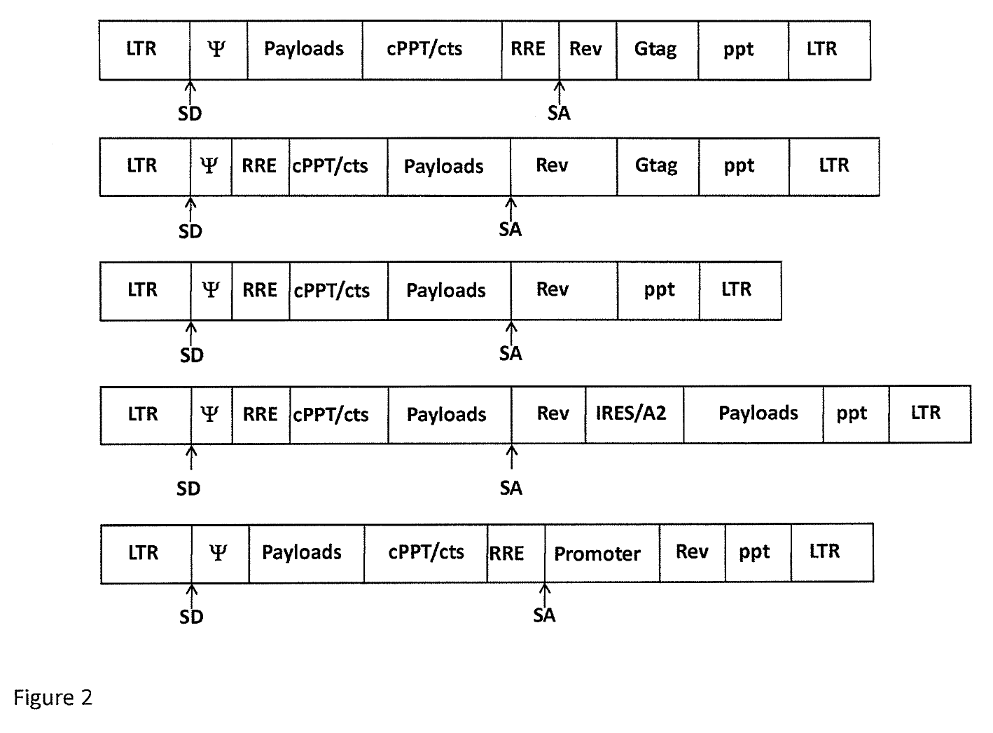

SEQ ID NO: 3 is an example of a DNA prime plasmid construct of FIG. 2A.

SEQ ID NO: 4 is an example of FIG. 2B.

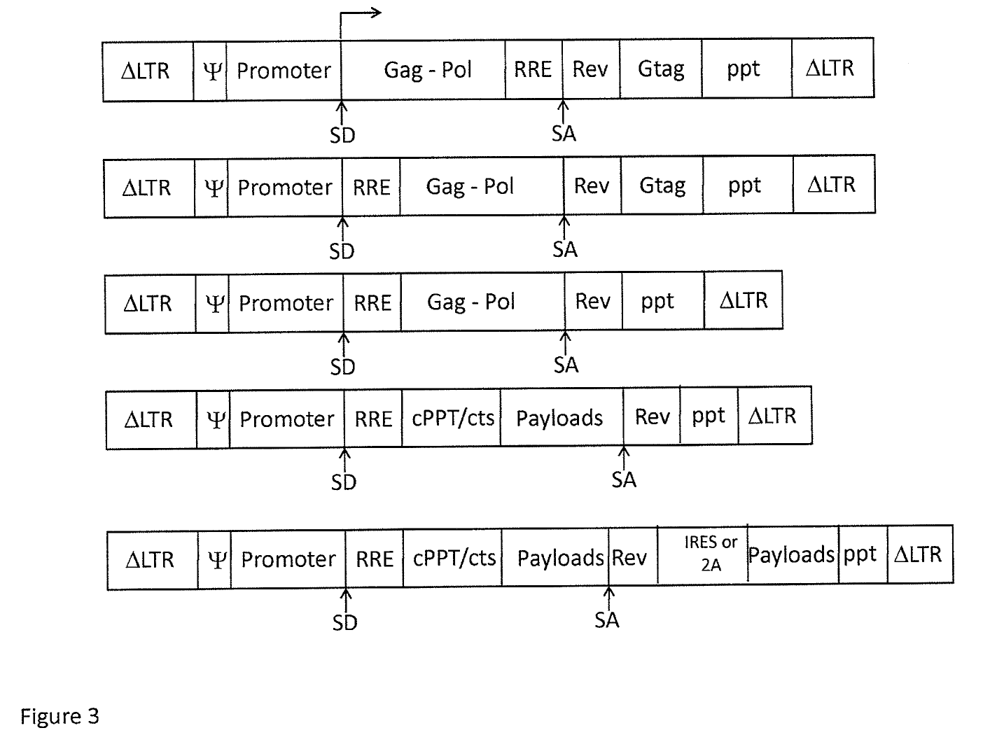

SEQ ID NO: 5 is an example of FIG. 3A.

SEQ ID NO: 6 is an example of FIG. 3B.

SEQ ID NO: 7 is an exemplary packaging signal sequence.

SEQ ID NO: 8 is an example of 5′ LTR of HIV of vector pNL4-3.

SEQ ID NO: 9 is an example of 3′ LTR of HIV of vector pNL4-3.

SEQ ID NO: 10 is an example of REV gene of HIV vector pNL4-3.

SEQ ID NO: 11 is an example of RRE of HIV vector pNL4-3.

SEQ ID NO: 12 is an example of minimum packaging sequence of HIV vector pNL4-3.

SEQ ID NO: 13 is an example of cPPT/cTS of HIV vector pNL4-3.

SEQ ID NO: 14 is an example of PPT of HIV vector pNL4-3.

SEQ ID NO: 15 is an example of a gag/pol coding sequence of HIV vector pNL4-3.

SEQ ID NO: 16 is an example of a frameshift modified gag/pol coding sequence having a frameshift mutation.

SEQ ID NO: 17 is an example of (GGT) codon for myristylation receptor glycine residue of Gag of HIV vector pNL4-3.

### DETAILED DESCRIPTION OF THE INVENTION

This invention relates to improved lentiviral based immunogenic vectors for use in prophylactic and therapeutic treatment of viral, parasitic, or bacterial infections, and cancer, methods of use thereof, and methods of their manufacture. This invention relates more particularly to a lentiviral vector, methods of making, modifying, propagating, packaging the same, methods of using such a vector and host cell, and, more particularly, for use with human immunodeficiency virus (HIV) infection and other diseases.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

As used herein, the term “vector” means a nucleic acid molecule (typically DNA or RNA) that serves to transfer a passenger nucleic acid sequence (i.e., DNA or RNA) into a host cell. Three common types of vectors include plasmids, phages and viruses. Preferably, the vector is a virus, which includes the encapsidated forms of vector nucleic acids, and viral particles in which the vector nucleic acids have been packaged. Transduction of cells with LV includes, but not limited to, three major steps: cell entry, conversion of vector RNA into DNA and delivering of DNA to nucleus. Transduction-competent (or maybe capable) LVs have all elements to accomplish all above-mentioned steps.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

As used herein, a “tissue-specific” promoter means a nucleotide sequence which, when operably linked with a polynucleotide which encodes or specifies a gene product, causes the gene product to be produced in a living human cell substantially only if the cell is a cell of the tissue type corresponding to the promoter.

Lentiviral Vectors

The present invention provides for improved lentiviral vectors for vaccine delivery. The vectors of the present invention comprise a 5′ LTR (SEQ ID NO: 8) and a 3′ LTR (SEQ ID NO: 9); a first nucleic acid sequence operably linked to said 5′ LTR, also referred to herein as the “payload”; and a second nucleic acid sequence, that is operably linked to said 5′ LTR comprising a functional REV (SEQ ID NO: 10) and an RRE (SEQ ID NO: 11)-containing sequence wherein the RRE-containing sequence is located upstream of the REV coding sequence, wherein transcription of said first nucleic acid sequence and said second nucleic acid sequence is driven by said 5′ LTR. “Payload” is that portion of the vector which is distinct from the packaging signal (SEQ ID NO: 7) required to package the RNA version of the lentiviral vector during viral production. SEQ ID NO: 12 is an example of a minimum packaging sequence.

The vector of the present invention further comprises a nucleic acid sequence encoding functionally active lentiviral RNA packaging elements (SEQ ID NO: 12). The full-length lentiviral RNA is selectively incorporated into the viral particles as a noncovalent dimer. RNA packaging into virus particles is dependent upon specific interactions between RNA and the nucleocapsid protein (NC) domain of the Gag protein. In nature, incorporation of the HIV genomic RNA into the viral capsid (referred to as “encapsidation”) involves the so-called Psi region located immediately upstream of the Gag start codon and folded into four stem-loop structures, is important for genome packaging; SL1 to SL4. In particular, SL1 contains the dimerization initiation site (DIS), a GC-rich loop that mediates in vitro RNA dimerization through kissing-complex formation, presumably a prerequisite for virion packaging of RNA. Additional cis-acting sequences have also been shown to contribute to RNA packaging. Some of these elements are located in the first 50 nucleotides (nt) of the Gag gene, including SL4, whereas others are located upstream of the splice-donor site (SD1), and are actually mapped to a larger region covering the first 350-400 nt of the genome, including about 240 nt upstream of SL1. The SL1-4 region is an example of a simple sequence essential for RNA packaging. Other such sequences are known by those of skill in the art.

The lentiviral vector also comprises a nucleic acid sequence encoding a functional central polypurine tract (cPPT)/cTS (SEQ ID NO: 13) and 3′ LTR proximal polypurine tract (PPT) (SEQ ID NO: 9). HIV and other lentiviruses, as are known in the art, have the unique property to replicate in non-dividing cells. This property relies on the use of a nuclear import pathway enabling the viral DNA to cross the nuclear membrane of the host cell. In HIV reverse transcription, a central strand displacement event consecutive to central initiation and termination of plus strand synthesis creates a plus strand overlap; the central DNA flap. This central DNA flap is a region of triplestranded DNA created by two discrete half-genomic fragments with a central strand displacement event controlled in cis by a central polypurine tract (cPPT) and a central termination sequence (CTS) during HIV reverse transcription. A central copy of the polypurine tract cis-active sequence (cPPT), present in all lentiviral genomes, initiates synthesis of a downstream plus strand. The upstream plus strand segment initiated at the 3′ PPT will, after a strand transfer, proceed until the center of the genome and terminate after a discrete strand displacement event. This last event of HIV reverse transcription is controlled by the central termination sequence (CTS).

It is an aspect of the present invention that the transcription of the payload as well as the Rev coding sequence and RRE-containing sequence is driven by the same promoter, i.e., the 5′ LTR (SEQ ID NO: 8). The 5′ LTR has sufficient basal activity to drive transcription of a payload comprising nucleic acids that encode full length antigenic sequences, as well as packaging sequences, and Rev coding sequence and RRE-containing sequence on the same transcription unit, with the proviso that the RRE-containing sequence is located upstream of the REV coding sequence. The 5′ LTR can be derived from various strains and clades of HIV, as are known in the art, and optimized for stronger basal promoter-like function. In particular, the 5′ LTR from HIV-1 Clade E can exhibit strong basal promoter activity. Various strains and clades of HIV are known in the art and may be used to generate the lentiviral vaccine vectors of the present invention including for example, without limitation, HIV-1 groups: M (for major) (A, B, C, D, E, F, G, H, I, and J), O (outlier or “outgroup”), which is a relatively rare group currently found in Cameroon, Gabon, and France, and a third group, designated N (new group), and any circulating recombinant forms thereof. The 5′ LTR further drives expression of the payload and Rev proteins. The HIV Rev protein directs the export of unspliced or partially spliced viral transcripts from the nucleus to the cytoplasm in mammalian cells. Rev contains the RNA binding domain, which binds the RRE present on target transcripts. Export activity is mediated by a genetically defined effector domain, which has been identified as a nuclear export signal.

In another embodiment of the present invention, the lentiviral vectors of the present invention can comprise at least one, but can optionally comprise two or more nucleotide sequences of interest. In order for two or more nucleotide sequences of interest to be expressed, there may be two or more transcription units within the vector genome, one for each nucleotide sequences of interest. In those instances, it is preferable to use one or more internal ribosome entry sites (IRESs) or FMDV 2A-like sequences for translation of the second (and subsequent) coding sequence(s) in a poly-cistronic (or as used herein, “multicistronic”) message (Adam et al 1991 J. Virol. 65, 4985, the entire contents of which are incorporated herein by reference). The IRES/2A(s) may be of viral origin (such as EMCV IRES, PV IRES, or FMDV 2A-like sequences) or cellular origin (such as FGF2 IRES, NRF IRES, Notch 2 IRES or EIF4 IRES). Non-limiting examples of lentiviral vector constructs of the present invention that utilize an IRES sequence may be found in FIGS. 2 and 3 infra.

It is an aspect of the first nucleic acid sequence to encode one or more antigenic or immunogenic sequences of interest. The antigenic or immunogenic sequence of interest may include a sequence or sequences that encode any protein or protein fragment that can elicit either a cell mediated or humoral response or both. The antigens can encode proteins or protein fragments of bacteria, viruses, and other microorganisms. In general, the present invention may be used to treat any viral infection or other infection associated with the desired antigenic or immunogenic sequence of interest.

In addition, in certain embodiments of the present invention, the first nucleotide sequence of interest or “payload” sequence can also includes those nucleotide sequences encoding enzymes, cytokines, chemokines, growth factors, hormones, antibodies, anti-oxidant molecules, engineered immunoglobulin-like molecules, a single chain antibody, fusion proteins, immune co-stimulatory molecules, immunomodulatory molecules, a transdominant negative mutant of a target protein, a toxin, a conditional toxin, an antigen, a tumour suppresser protein and growth factors, membrane proteins, pro- and anti-angiogenic proteins and peptides, vasoactive proteins and peptides, anti-viral proteins and derivatives thereof (such as with an associated reporter group). The nucleotide sequences of interest may also encode pro-drug activating enzymes. When used in a research context, the nucleotide sequences of interest may also encode reporter genes such as, but not limited to, green fluorescent protein (GFP), luciferase, .beta.-galactosidase, or resistance genes to antibiotics such as, for example, ampicillin, neomycin, bleomycin, zeocin, chloramphenicol, hygromycin, kanamycin, among others. The nucleotide sequences of interest may also include those which function as anti-sense RNA, small interfering RNA (siRNA), or ribozymes, or any combination thereof.

The antigenic sequence of the present invention may further comprise any tumor antigens as are known in the art. A tumor antigen is a protein or protein or peptide fragment thereof produced in tumor cells that triggers an immune response in the host. Tumor antigens are useful in identifying tumor cells and are potential candidates for use in cancer therapy. Tumor antigens may include alphafetoprotein, carcinoembryonic antigen, CA-125, epithelial tumor antigen, tyrosinase, melanoma associated antigen, by way of example. Also, another aspect of the invention is antigenic compounds, proteins and protein fragments associated with Alzheimer's disease.

The vector further comprises expression of said one or more sequences of interest which is dependent upon REV-RRE activity. It is a feature of the invention that Rev and RRE as expressed by the same 5′ LTR as the payload are essential to the transport of the payload to the cytoplasm after translation in the cell nucleus. Rev is a small regulatory protein of HIV that is essential for virus replication. The biological role of Rev is to control the pattern of viral gene expression by promoting the transition from the early phase of infection, during which small regulatory proteins are expressed, to the late stage, when larger structural proteins are synthesized and assembled into viral particles. Rev binds to a highly structured RNA region, the Rev response element (RRE), where it forms an oligomeric ribonucleoprotein complex with about 8 to 10 Rev proteins binding to the RRE. The most important region of the RRE consists of five stem-loop domains radiating from a central junction, with the core element for Rev binding consisting of a stem loop-structure containing a purine rich bulge region (SEQ ID NO: 11).

In one embodiment of the invention, the first nucleic acid sequence of the invention comprises a gag/pol coding sequence (SEQ ID NO: 15). The invention may encode full-length gag/pol sequences or parts or derivatives thereof. The first nucleic acid sequence whether it encode gag/pol or any other antigenic sequence of the claimed invention may include modified or unmodified sequences. (SEQ ID NO: 15). The lentiviral vector comprises the cPPT as a part of a pol coding sequence of the gag/pol coding sequence. When other payloads other than gag/pol are in the vector, cPPT, PPT and CTS are required in the vectors as discussed in other embodiments disclosed herein which do not include gag/pol coding sequence as the antigenic sequence of interest.

It is an embodiment of the invention wherein said gag/pol encoding sequence is modified to increase cellular expression through inclusion of a modified gag coding sequence in the lentiviral vector construct. The inclusion of the pol gene in the form of the gag/pol precursor in our current vaccine is most likely suboptimal with regard to inefficient expression of the Pol antigen. The expression levels of the Pol protein generally are low during natural infection because of the frameshift required for translation of pol coding sequences. This form of Pol expression results in an up to 95% reduction in Pol protein compared to Gag. To increase Pol expression, the frameshift between gag and pol can be removed, resulting in equimolar or nearly equimolar expression of Gag and Pol whereas the secretion of virus-like particles (VLP) will be impaired. As such, the vector comprises a frameshift within the gag sequence in order to optimize expression (SEQ ID NO: 16).

In an embodiment of the invention, the lentiviral vector also supports a high level of expression of gag-pol fusion protein not mediated by translational frameshifting. The invention further comprises at least one nucleotide substitution which alters the myristylation receptor glycine residue of Gag (SEQ ID NO: 17). Myristylation is a key component in the HIV particle assembly process. Gag p55 undergoes posttranslational myristylation at the N-terminus of matrix, which allows the Gag complex to attach to the cell membrane via the myristic acid moiety. Disruption of the myristylation site results in an accumulation of Gag proteins in the cytoplasm. The higher concentration of proteins within the cytoplasm most likely increases proteosomal degradation of these polypeptides into the endogenous antigen pathway, resulting in the loading of Gag peptides on MHC class 1 molecules leading to higher immune responses.

The level of antigen expression can be increased by using codon optimized genes as well. Synthetic genes, created with the codon bias found in highly expressed human genes, have been shown to be much more efficiently expressed in human cells and to elicit higher and more reproducible levels of immune responses than did native coding sequences. Another implication of over-expression is that the amount of a lentiviral vector or DNA prime that needs to be administered to elicit an immune response can be greatly reduced.

In another embodiment of the present invention, the lentiviral vector of the present invention may include, without limitation, those lentiviruses can be divided into viruses that infect primate (HIV-1, HIV-2, simian immunodeficiency virus (SIV)) and non-primate (feline immunodeficiency virus (FIV), equine infectious anemia virus (EIAV), Bovine Immunodeficiency Virus (BIV), caprine arthritis encephalitis virus (CAEV), visna maedi virus (VV), Jembrana disease virus (JDV)).

In yet another embodiment of the present invention, the lentiviral vector of the present invention could be also modified by removing the transcriptional elements of HIV LTR; such as in a so-called self-inactivating (SIN) vector configuration. The modalities of reverse transcription, which generates both U3 regions of an integrated provirus from the 3′ end of the viral genome, facilitate this task by allowing the creation of so-called self-inactivating (SIN) vectors. Self-inactivation relies on the introduction of a disruption (employing for example, deletion, mutation and element insertion) in the U3 region of the 3′ long terminal repeat (LTR) of the DNA used to produce the vector RNA. During reverse transcription, this deletion is transferred to the 5′ LTR of the proviral DNA. If enough sequence is eliminated to abolish the transcriptional activity of the LTR, the production of full-length vector RNA in transduced cells is abolished. This minimizes the risk that RCRs will emerge. Furthermore, it reduces the likelihood that cellular coding sequences located adjacent to the vector integration site will be aberrantly expressed, either due to the promoter activity of the 3′ LTR or through an enhancer effect. Finally, a potential transcriptional interference between the LTR and the internal promoter driving the transgene is prevented by the SIN design. One example of a SIN based lentiviral vector is described in U.S. Pat. No. 6,924,144, the entire contents of which are incorporated herein by reference in its entirety. Non-limiting representative examples of SIN-based lentiviral vectors of the present invention may be generated from one or more of the constructs specifically shown in FIGS. 1, 2, and/or 3 described herein or any combination thereof.

In yet another embodiment, the invention includes a pharmaceutical composition comprising the lentiviral vector described herein above comprising: a 5′ LTR and a 3′ LTR; a first nucleic acid sequence operably linked to said 5′ LTR; and a second nucleic acid sequence operably linked to said 5′ LTR comprising a functional REV coding sequence and a rev response element (RRE)-containing sequence wherein the RRE-containing sequence is located upstream of the REV coding sequence, wherein transcription of said first nucleic acid sequence and said second nucleic acid sequence is driven by said 5′ LTR; and further comprising a “pharmaceutically acceptable carrier” or “genetic adjuvant.” “Pharmaceutically acceptable carriers” include, without limitation, PBS, buffers, water, TRIS, other isotonic solutions or any solution optimized to not damage the viral components of the vector.

The above described lentiviral vectors can be introduced into a host cell for the prophylactic and therapeutic treatment of viral infection, as well as for other reasons described herein. Accordingly, the present invention provides a host cell comprising a vector according to the invention. The isolation of host cells, and/or the maintenance of such cells or cell lines derived therefrom in culture, has become a routine matter and one in which the ordinary skilled artisan is well versed.

A “host cell” can be any cell, and, preferably, is a eukaryotic cell. Desirably, the host cell is an antigen presenting cell. Such a cell includes, but is not limited to, a skin fibroblast, a bowel epithelial cell, an endothelial cell, an epithelial cell, a dendritic cell, a plasmacytoid dendritic cell, Langerhan's cells, a monocyte, a mucosal cell and the like. Preferably, the host cell is of a eukaryotic, multicellular species (e.g., as opposed to a unicellular yeast cell), and, even more preferably, is a mammalian, e.g., human cell.

A cell can be present as a single entity, or can be part of a larger collection of cells. Such a “larger collection of cells” can comprise, for instance, a cell culture (either mixed or pure), a tissue (e.g., endothelial, epithelial, mucosa or other tissue), an organ (e.g., lung, liver, muscle and other organs), an organ system (e.g., circulatory system, respiratory system, gastrointestinal system, urinary system, nervous system, integumentary system or other organ system), or an organism (e.g., a bird, mammal, or the like). Preferably, the organs/tissues/cells being targeted are of the circulatory system (e.g., including, but not limited to blood, including white blood cells), the mucosal system of the nose, trachea, bronchi, bronchioles, lungs, and the like), gastrointestinal system (e.g., including mouth, pharynx, esophagus, stomach, intestines, salivary glands, pancreas, liver, gallbladder, and others), urinary system (e.g., such as kidneys, ureters, urinary bladder, urethra, and the like), nervous system (e.g., including, but not limited to, brain and spinal cord, and special sense organs, such as the eye) and integumentary system (e.g., skin, epidermis, and cells of subcutaneous or dermal tissue). Even more preferably, the cells being targeted are selected from the group consisting of antigen presenting cells. The target cells need not be normal cells and can be diseased cells. Such diseased cells can be, but are not limited to, tumor cells, infected cells, genetically abnormal cells, or cells in proximity or contact to abnormal tissue such as tumor vascular endothelial cells.

A “vector” is a nucleic acid molecule (typically DNA or RNA) that serves to transfer a passenger nucleic acid sequence (i.e., DNA or RNA) into a host cell. Three common types of vectors include plasmids, phages and viruses. Preferably, the vector is a virus, which includes the encapsidated forms of vector nucleic acids, and viral particles in which the vector nucleic acids have been packaged. Transduction of cells with LV includes, but not limited to, three major steps: cell entry, conversion of vector RNA into DNA and delivering of DNA to nucleus. Transduction-competent (or maybe capable) LVs have all elements to accomplish all above-mentioned steps.

The vector is not a wild-type strain of a virus, inasmuch as it comprises human-made mutations or modifications. Thus, the vector typically is derived from a wild-type viral strain by genetic manipulation (e.g., by addition, deletion, mutation, insertion or other techniques known in the art) to comprise lentivirus, as further described herein. As encompassed herein, a vector can comprise either DNA or RNA. For instance, either a DNA or RNA vector can be used to derive the virus. Similarly, a cDNA copy can be made of a viral RNA genome. Alternatively, a cDNA (or viral genomic DNA) moiety can be transcribed in vitro to produce RNA. These techniques are well-known to those skilled in the art, and also are described.

In one aspect, the invention provides for a method for inducing immune responses in a subject said method comprising administering a pharmaceutical composition comprising the lentiviral vector disclosed herein. The method according to the invention provides for expression of the one or more genes of interest to potentiate or induce immunity. The immunity that is induced can be cellular or humoral immunity. More particularly, the cellular immune response can potentiate activity of CD8+ and/or CD4+ cells.

It is yet another embodiment of the invention to inhibit the replication of an infective replicative human immunodeficiency virus (HIV) in a cell comprising: A method for inducing immune responses in a subject said method comprising administering a pharmaceutical composition comprising a lentiviral vector comprising a 5′ LTR and a 3′ LTR; a first nucleic acid sequence operably linked to said 5′ LTR; and a second nucleic acid sequence operably linked to said 5′ LTR comprising a functional REV coding sequence and a rev response element (RRE)-containing sequence wherein the RRE-containing sequence is located upstream of the REV coding sequence, wherein transcription of said first nucleic acid sequence and said second nucleic acid sequence is driven by said 5′ LTR.

The method further comprises sequentially contacting said cell with a second lentiviral vector expressing the same first nucleic acid as the first lentiviral vector. The method can also be used with a prime/boost approach wherein the first vector is not required to be a lentiviral vector so long as the prime comprises the same payload or genes of interest as the second lentiviral vector which is administered subsequently.

It is yet another embodiment of the method of the invention wherein the lentiviral vector can be administered as a prime/multiple boost protocol.

It is yet another embodiment of the method of the invention wherein the lentiviral vector can be administered as a prime/boost or prime/multiple boost protocol with alternate pseudotyping envelopes such as VSV-G or baculovirus gp-64 proteins or a combination thereof as described infra.

It is yet another embodiment of the invention to enhance immune response in a subject comprising: contacting a cell with a vector comprising: a 5′ LTR and a 3′ LTR; a first nucleic acid sequence operably linked to said 5′ LTR; and a second nucleic acid sequence operably linked to said 5′ LTR comprising a functional REV coding sequence and a rev response element (RRE)-containing sequence wherein the RRE-containing sequence is located upstream of the REV coding sequence, wherein transcription of said first nucleic acid sequence and said second nucleic acid sequence is driven by said 5′ LTR.

The method further comprises sequentially contacting said cell with a second lentiviral vector expressing the same first nucleic acid as the first lentiviral vector. The method can also be used with a prime/boost or prime/multiple boost approach wherein the first vector is not required to be a lentiviral vector so long as the prime comprises the same payload or genes of interest as the second lentiviral vector which is administered subsequently. Subsequent administration can be at two weeks, four weeks, one month, two months, six months, or one year and as appropriate regimens to maintain immune response over time.

It is yet another embodiment of the invention to provide for a prime/boost approach for increasing the immunogenicity of a vector in a host comprising administering a pharmaceutical composition comprising a lentiviral vector comprising a 5′ LTR and a 3′ LTR; a first nucleic acid sequence operably linked to said 5′ LTR; and a second nucleic acid sequence operably linked to said 5′ LTR comprising a functional REV coding sequence and a rev response element (RRE)-containing sequence wherein the RRE-containing sequence is located upstream of the REV coding sequence, wherein transcription of said first nucleic acid sequence and said second nucleic acid sequence is driven by said 5′ LTR, wherein the immunogenicity of said vector is increased due to the prime/boost or prime/multiple boost protocol. The method further comprises sequentially administering said host with a second lentiviral vector expressing the same first nucleic acid as the first lentiviral vector. “Immunogenicity” or “potentiation” is the ability of the vector to induce a cellular (i.e., cell mediated) and/or humoral immune response against the transgene that can be systemic and/or a mucosal response. “Cellular immunity” is an immune response that does not involve antibodies but involves the activation of macrophages, natural killer cells or lymphocytes (i.e. CD8+ or CD4+ activation) and the release of cytokines or other mediators in response to an antigen. Humoral immunity is an antibody mediated immune response.

In yet another aspect of the present invention, a method of propagating and selectively packaging a lentiviral vector of the present invention is provided. The method comprises cells, cell lines and methods as are known in the art and more particularly described in commonly owned applications Application No. 60/585,464, Jul. 1, 2004, application Ser. No. 11/172,147, filed Jun. 30, 2005, U.S. Pat. No. 6,835,568, and US Publication Number 20030026791, the text of each of these recited applications, patent, and published application are incorporated herein by reference in their entirety as if fully set forth herein.

In yet another embodiment of the present invention, the lentiviral vector vaccine constructs of the present invention further comprise those lentiviral vectors in which the lentiviral integrase function has been deleted and/or abrogated by site directed mutagenesis. Insertional mutagenesis has been observed in clinical trials with oncoretroviral vectors and this has prompted detailed study of genotoxicty of all integrating vectors. The most straightforward approach for several vaccine applications would be avoiding the possibility of integration. Non-integrating lentiviral vectors have been developed by mutating the integrase gene or by modifying the attachment sequences of the LTRs. In particular, among the mutations studied, the D64V substitution in the catalytic domain has been frequently used because it shows the strong inhibition of the integrase without affecting proviral DNA synthesis. It has been reported that the mutation allows a transduction efficiency only slightly lower than integrative vectors but a residual integration that is about 1000-fold lower than an integrative vector at low vector doses. Another mutation described, D116N, resulted in residual integration about 2000 times lower than control vectors. In a couple of instances it has been shown that a single administration of an integrase (IN)-defective SIN LV elicits a significant immune response in the absence of vector integration and may be a safe and useful strategy for vaccine development. Thus, specifically contemplated within the scope of this invention is the modification to render the lentiviral vectors able to exist in episomal form yet still being able to provide transgene expression.

The lentiviral vector can be compatible with the host cell into which it is introduced, i.e., it is capable of imparting expression on the cell of the vector-encoded nucleic acid sequences. A coding sequence is “operably linked” to a promoter when the promoter is capable of directing transcription of the coding sequence.

“Pseudotyping” a virion is accomplished by co-transfecting a packaging cell with both the lentiviral vector of interest and a helper vector encoding at least one envelope protein of another virus or a cell surface molecule (see, for example, U.S. Pat. No. 5,512,421, the entire text of which is herein incorporated by reference in its entirety). One viral envelope protein commonly used to pseudotype lentiviral vectors is the vesicular stomatitis virus-glycoprotein G (VSV-G), which is derived from a rhabdovirus. Other viral envelopes proteins that may be used include, for example, rabies virus-glycoprotein G and baculovirus gp-64. The use of pseudotyping broadens the host cell range of the lentiviral vector particle by including elements of the viral entry mechanism of the heterologous virus used. Pseudotyping of lentiviral vectors with, for example, VSV-G for use in the present invention results in lentiviral particles containing the lentiviral vector nucleic acid encapsulated in a nucleocapsid which is surrounded by a membrane containing the VSV-G envelope protein. The nucleocapsid preferably contains proteins normally associated with the lentiviral vector. The surrounding VSV-G protein containing membrane forms part of the viral particle upon its egress from the producer cell used to package the lentiviral vector. In an embodiment of the invention, the lentiviral particle is derived from HIV and pseudotyped with the VSV-G protein. Pseudotyped lentiviral particles containing the VSV-G protein can infect a diverse array of cell types with higher efficiency than amphotropic viral vectors. The range of host cells includes both mammalian and non-mammalian species, such as humans, rodents, fish, amphibians and insects.

Even though VSV-G pseudotyping has been described as being the most efficient for cutaneous transduction, a great advantage of using LV is that it is possible to target the vector to specific tissues or cells by replacing and/or modifying the virion envelope. LVs are remarkably compatible with a broad range of viral envelope glycoproteins providing them with added flexibility; Rabies, Mokola, LCMV, Ross River, Ebola, MuLV, Baculovirus GP64, HCV, Sindai virus F protein, Feline Endogenous Retrovirus RD114 modified, Human Endogenous Retroviruses, Seneca virus, GALV modified and HA influenza glycoproteins or a combination thereof, to name a few of those viral envelope glycoproteins explored. In addition to modification or replacement of the entire envelope, flexibility of LV platform for targeting different cell types was further demonstrated by refining the surface of LV particles via the display of cell-specific ligands. For vaccine applications, VSV-G as a pseudotyping envelope confers some important advantages, such as a broad cellular tropism (including dendritic cells) and low preexisting immunity in the human population. VSV-G could eventually be replaced by other envelopes if needed, for example in the case of multiple vector administration, although anti-VSV-G immunity does not seem to prevent repeated vector administrations.

Such pseudotyped lentiviral vectors are further characterized in that they can comprise a 5′ LTR from a lentivirus; a 3′ LTR from a lentivirus; a splice donor site 3′ of the 5′ LTR; a first nucleic acid sequence operably to the 5′ LTR; a RRE 3′ of the first nucleic acid sequence; a splice acceptor site 3′ of the RRE; a heterologous promoter 3′ of the RRE; a splice acceptor site 3′ of the RRE; and a Rev gene 3′ of splice acceptor site. Once again, the functional REV coding sequence and a rev response element (RRE)-containing sequence wherein the RRE-containing sequence is located upstream of the REV coding sequence, wherein transcription of said first nucleic acid sequence and said second nucleic acid sequence is driven by said 5′ LTR. The first nucleic acid sequence encodes the full length wild-type sequence of either one or both of the Gag and Pol proteins.

In certain embodiments of the present invention, the heterologous promoter comprises viral, human, and/or synthetic promoters or a combination thereof. In one embodiment, heterologous viral promoters comprise Mouse Mammary Tumor Virus (MMTV) promoter, Moloney virus, avian leukosis virus (ALV), Cytomegalovirus (CMV) immediate early promoter/enhancer, Rous Sarcoma Virus (RSV), adeno-associated virus (AAV) promoters; adenoviral promoters, and Epstein Barr Virus (EBV) promoters, or any combination thereof. In one embodiment, heterologous human promoters comprise Apolipoprotein E promoter, Albumin promoter, Human ubiquitin C promoter, human tissue specific promoters such as liver specific promoter, prostate specific antigen (psa) promoter, Human phosphoglycerate kinase (PGK) promoter, Elongation factor-1 alpha (EF-1a) promoter, dectin-2 promoter, HLA-DR promoter, Human CD4 (hCD4) promoter, or any combination thereof. In yet another embodiment, the synthetic promoters comprise those promoters described in U.S. Pat. No. 6,072,050, the contents of which are incorporated by reference in its entirety.

It is yet another aspect of the invention that various elements of the construct may include functional equivalent derivatives, fragments or modifications thereof that have been engineered into the nucleotide coding sequences and/or amino acid sequences of the first nucleic acid sequence or the second nucleic acid sequence of the lentiviral vectors of the present invention. Any and all such nucleotide variations and resulting amino acid polymorphisms or variations that are the result of natural genotypic, allelic variation, or that have been artificially engineered, and that do not alter the functional activity are intended to be within the scope of the invention. Thus, functional equivalent derivatives, fragments or modifications thereof of the first nucleic acid sequence or the second nucleic acid sequence of the lentiviral vectors of the present invention can be created by introducing one or more nucleotide substitutions, additions or deletions into the nucleotide sequence of marker nucleic acids, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

Thus, in one embodiment, the present invention comprises a packaging signal sequence as described in SEQ ID NO: 7 and functionally active derivatives, fragments or mutations thereof wherein the derivatives, fragments or mutations can be from about 60% to about 100% homologous to SEQ ID NO: 7.

In another embodiment, the present invention comprises a 5′ LTR of HIV vector pNL4-3 as described in SEQ ID NO: 8 and functionally active derivatives, fragments or mutations thereof wherein the derivatives, fragments or mutations can be from about 60% to about 100% homologous to SEQ ID NO: 8.

In another embodiment, the present invention comprises a 3′ LTR of HIV vector pNL4-3 as described in SEQ ID NO: 9 and functionally active derivatives, fragments or mutations thereof wherein the derivatives, fragments or mutations can be from about 60% to about 100% homologous to SEQ ID NO: 9.

In another embodiment, the present invention comprises a REV gene of HIV vector pNL4-3 as described in SEQ ID NO: 10 and functionally active derivatives, fragments or mutations thereof wherein the derivatives, fragments or mutations can be from about 60% to about 100% homologous to SEQ ID NO: 10.

In another embodiment, the present invention comprises a sequence of functional RRE of HIV vector pNL4-3 as described in SEQ ID NO: 11 and functionally active derivatives, fragments or mutations thereof wherein the derivatives, fragments or mutations can be from about 60% to about 100% homologous to SEQ ID NO: 11.

In another embodiment, the present invention comprises a minimum packaging sequence of HIV vector pNL4-3 as described in SEQ ID NO: 12 and functionally active derivatives, fragments or mutations thereof wherein the derivatives, fragments or mutations can be from about 60% to about 100% homologous to SEQ ID NO: 12.

In another embodiment, the present invention comprises cPPT/cTS of HIV vector pNL4-3 as described in SEQ ID NO: 13 and functionally active derivatives, fragments or mutations thereof wherein the derivatives, fragments or mutations can be from about 60% to about 100% homologous to SEQ ID NO: 13.

In another embodiment, the present invention comprises PPT of HIV vector pNL4-3 as described in SEQ ID NO: 14 and functionally active derivatives, fragments or mutations thereof wherein the derivatives, fragments or mutations can be from about 60% to about 100% homologous to SEQ ID NO: 14.

In another embodiment, the present invention comprises a gag/pol coding sequence of HIV vector pNL4-3 as described in SEQ ID NO: 15 and functionally active derivatives, fragments or mutations thereof wherein the derivatives, fragments or mutations can be from about 60% to about 100% homologous to SEQ ID NO: 15.

In another embodiment, the present invention comprises a frameshift modified gag/pol fusion coding sequence of HIV vector pNL4-3 as described in SEQ ID NO: 16 and functionally active derivatives, fragments or mutations thereof wherein the derivatives, fragments or mutations can be from about 60% to about 100% homologous to SEQ ID NO: 16.

It is to be understood that the compositions of the present invention may contain more than one vectors, which may be the same or different, in order to prophylactically or therapeutically treat the progression of various subtypes of human immunodeficiency virus infection after vaccination. Such combinations may be selected according to the desired immunity. When a lentiviral based vaccine is administered to an animal or a human, it can be combined with one or more genetic adjuvants as are known to one of ordinary skilled in the art. “Genetic adjuvants” can be inserted as payloads in the lentiviral vector of the present invention to increase or enhance the immune response elicited by expression of the antigenic sequence of interest of the present invention. These genetic adjuvants can be on different constructs but co-expressed or on the same vector construct yet contained in different expression cassettes. Such desirable genetic adjuvants may include, without limitation, toll like receptors, CpG, cytokines (e.g., the interleukins (ILs) IL-1.beta., Il-2, Il-10, Il-12, Il-15, CTA1-DD, fas antigen, flagellin, etc.), or combinations thereof. Other desirable genetic adjuvants include, without limitation, the DNA sequences encoding GM-CSF, the interferons (IFNs) (for example, IFN-.alpha., IFN-.beta., and IFN-.gamma.), TNF-.alpha., and combinations thereof. The genetic adjuvants may also be a immunostimulatory polypeptide from Parapox virus, such as a polypeptide of Parapox virus strain D1701 or NZ2 or Parapox immunostimulatory polypeptides B2WL or PP30 (see, e.g., U.S. Pat. No. 6,752,995). Still other such biologically active factors and immunomodulators or immunostimulants that are known in the art that enhance the antigen-specific immune response may be readily selected by one of skill in the art, and a suitable plasmid vector containing the same factors constructed by known techniques.

Methods of Use

The lentiviral vectors of the present invention can be used to treat a wide array of viral infections caused by various viruses including, but not limited to, viruses from the groups including dsDNA viruses (e.g. adenoviruses, herpesviruses, etc.); ssDNA viruses (e.g. parvoviruses); dsRNA viruses (e.g. reoviruses, rotavirus); (+)ssRNA viruses (e.g. picornavirus, togavirus etc); (−)ssRNA viruses (e.g. Orthomyxoviruses, Rhabdoviruses, etc.); ssRNA-RT viruses (e.g. Retroviruses, HIV, SIV, etc.); and dsDNA-RT viruses (e.g. Hepadnaviruses).

The lentiviral vectors of the present invention can be used to treat disease cause by viruses from a number of viral families including, but not limited to, Herpes Simplex Virus (HSV)-1 (oral herpes simplex), HSV-2 (genital herpes simplex), Varicella Zoster virus (VZV) (chickenpox), Epstein-Barr virus (EBV) (mononucleosis), Cytomegalovirus (CMV) (TOXplasmosis, Rubella, Herpes simplex), Pox virus (smallpox), and other viruses as are known in the art.

The lentiviral vectors of the present invention can also be used to prophylactically or therapeutically treat various diseases including parasitic and microorganism ailments including, but not limited to, the disease malaria caused by mosquito transmission of the malarial parasite plasmodium to humans. The disease is caused by protozoan parasites of the genus Plasmodium. Only four types of the plasmodium parasite can infect humans. The most serious forms of the disease are caused by Plasmodium falciparum and Plasmodium vivax, but other related species (Plasmodium ovale, Plasmodium malariae) can also affect humans. Other diseases that can be treated by the method of the present invention are malaria, St. Louis encephalitis, dengue fever, yellow fever, West Nile virus; bubonic plague; Lyme disease, rocky mountain spotted fever, encephalitis; hantavirus; Chagas Disease, by way of example, not limitation. The lentiviral vector of the present invention can be modified to include antigens from any of the above viruses in the payload to therapeutically treat or as prophylaxis.

Also, treatable by the lentiviral vector of the methods of the present invention are bacterial infections including, but not limited to, the following: human disease causing bacteria of the order Spirochaetales including T. pallidum (Syphilis), S. pilosicoli (diarrhoea in humans especially in developing countries and in HIV patients), B. burgdorferi: (Lyme disease and related disorders), bacteria of the family Spirillaceae including C. jenuni (colitis, diarrhoea, enteritis, enterocolitis, gastroenteritis, etc.), H. pylori (gastritis, duodenal and peptic ulcers, Taynaud's phenomenon, etc.); bacteria of the Family Spirosomaceae including Pseudomonas (cellulitis, cerebrospinal fluid shunt infections, acute cystitis, endocarditis, chronic eye infections, neonatal meningitis, peritonitis, pneumonia), of the family Legionellaceae including Legionella (pneumonia in humans (legionellosis, legionnaire's disease, Pittsburgh pneumonia, nonpneumonic Pontiac fever, rhabdomyolysis, bacteraemia and septicemia, endocarditis, systemic infections in cell-mediated immunity disorders); bacteria Rhizobiacea including Agrobacterium (peritonitis in continuous ambulatory peritoneal dialysis); Achromobacter (postoperative and posttraumatic wound infectious complications); bacteria Shigella (watery diarrhea, dysentery); bacteria Salmonella (enteric fever, typhoid, paratyphoid, gastroenteritis, food poisoning, diarrhoea and/or vomiting) and other disease causing bacteria as are known to those of skill in the art.

The lentiviral vector of the present invention can be used as a cancer vaccine for the prophylactic and therapeutic treatment of cancer. Cancer vaccines are intended either to therapeutically or prophylactically (e.g. Gardisil®) reduce the burden of cancer. Treatment or therapeutic vaccines are administered to cancer patients and are designed to strengthen the body's natural defenses against cancers that have already developed. These types of vaccines can reduce the further growth of existing cancers, decrease the recurrence of treated cancers, or alter disease progression in cancer cells not killed by prior treatments. Prophylactic vaccines, on the other hand, are administered to healthy individuals and are designed to target cancer-causing viruses as prophylaxis to viral infection. Cancer vaccines used to treat cancers take advantage of antigens presented to stimulate the immune system to make a specific immune response. The lentiviral vaccine of the present invention has a two-fold benefit in that the lentiviral vector vaccine itself can stimulate the immune response as can the antigen sequence once expressed in the host cell.

The immune system generally does not recognize tumors as dangerous or foreign, and does not mount a strong immune response against them. One reason tumor molecules do not stimulate an effective immune response as thought by those of skill in the art to be that tumor cells are derived from normal cells. Therefore, even though there are many molecular differences between normal cells and tumor cells, cancer antigens are not truly foreign to the body, but are normal molecules, altered in subtle ways. Another reason tumors may not stimulate an immune response is that cancer cells have developed ways to “escape” from the immune system. Scientists now understand some of these modes of escape, which include shedding tumor antigens, and reducing the number of molecules and receptors that the body normally relies on to activate T cells (specific immune cells) and other immune responses. Reducing these molecules makes the immune system less responsive to the cancer cells; the tumor becomes less “visible” to the immune cells. Scientists hope that this knowledge can be used by researchers to design more effective vaccines. The lentiviral vector of the present invention can heighten the immune response and express cancer-related antigen to therapeutically treat the cancer.

Thus, in another embodiment, the lentiviral vector of the present invention further comprises the nucleic acid sequence encoding tumor antigen to produce the cancer antigen proteins.

Cancer cell antigens may be unique to individual tumors, shared by several tumor types, or expressed by the normal tissue from which a tumor grows. Cancer related antigens include but are not limited to: Prostate Specific Antigen (PSA) which is a prostate-specific protein antigen that can be found circulating in the blood, as well as on prostate cancer cells, the quantity of PSA rises when prostate cancer develops. Sialyl Tn (STn) which is a small, synthetic carbohydrate that mimics the mucin molecules (the primary molecule present in mucus) found on certain cancer cells; Heat Shock Proteins (HSPs) (e.g., gp96, etc.) which are produced in cells in response to heat, low sugar levels and other stress signals and in addition to protecting against stress, these molecules are also involved in the proper processing, folding, and assembling of proteins within cells. Ganglioside molecules (e.g., GM2, GD2, and GD3) which are complex molecules containing carbohydrates and fats and when ganglioside molecules are incorporated into the outside membrane of a cell and is a molecule expressed on the cell surface of a number of human cancers (GD2 and GD3 contain carbohydrate antigens expressed by human cancer cells); Carcinoembryonic antigen (CEA) which is found in high levels on tumors in people with colorectal, lung, breast and pancreatic cancer as compared with normal tissue and is thought to be released into the bloodstream by tumors and patients have been shown to mount T-cell responses to CEA; MART-1 (also known as Melan-A), which is an antigen expressed by melanocytes and is a specific melanoma cancer marker that is recognized by T cells and is more abundant on melanoma cells than normal cells; Tyrosinase, which is a key enzyme involved in the initial stages of melanin production, and studies have shown that tyrosinase is a specific marker for melanoma and is more abundant on melanoma cells than normal cells; and viral proteins on the outside coat of cancer-causing viruses are commonly used as antigens to stimulate the immune system to prevent infections with the viruses; and other cancer antigens are known to the skilled artisan.

An aspect of the invention is to combine the lentiviral vector with an adjuvant to heighten the immune response to cancer antigens, researchers usually attach a decoy substance, or adjuvant, that the body will recognize as foreign. Adjuvants are weakened proteins or bacteria which “trick” the immune system into mounting an attack on both the decoy and the tumor cells. Adjuvants of the present invention include, but are not limited to, Keyhole limpet hemocyanin (KLH) which is a protein made by a shelled sea creature found along the coast of California and Mexico known as a keyhole limpet and is a large protein that both causes an immune response and acts as a carrier for cancer cell antigens, since cancer antigens often are relatively small proteins that may be invisible to the immune system it provides additional recognition sites for immune cells known as T-helper-cells and may increase activation of other immune cells known as cytotoxic T-lymphocytes (CTLs); Bacillus Calmette Guerin (BCG) which is an inactivated form of the tuberculosis bacterium, is added to some cancer vaccines to boost the immune response to the vaccine antigen; Interleukin-2 (IL-2) (and other cytokines as are known in the art) which is/are protein made by the body's immune system that may boost the cancer-killing abilities of certain specialized immune system cells called natural killer cells; Granulocyte Monocyte-Colony Stimulating Factor (GM-CSF) which is a protein that stimulates the proliferation of antigen-presenting cells; QS21 which is a plant extract that, when added to some vaccines, can improve the body's immune response; Montanide ISA-51 is an oil-based liquid to boost an immune response.

The adjuvant may be co-administered with the vector of the present invention, sequentially administered prior to or subsequent to administration of the lentiviral vector of the present invention or, if appropriate, as a genetic adjuvant, as disclosed herein, to enhance the immune response in a patient.

The lentiviral vectors of the invention can carry the full-length Gag, Pol, a payload and Rev and RRE genes from HIV under the dependence of the native 5′ LTR. Optimization of the vaccine included route optimization in order to elicit both systemic and mucosal responses in mice. Protocol optimization was also performed by combining the lentiviral vector with other viral vectors or DNA plasmids, or both. Some of these protocols elicited broad and sustained immunogenicity. All studies were performed in mice and anti-HIV immunogenicity was assessed by Intracellular Cytokine Staining (ICS), Elispot, Tetramer staining, cytotoxicity, T-cell proliferation, and Elisa. A DNA prime/lentiviral vector boost protocol elicited high level anti-HIV humoral and cellular immunity (for example, and not by way of limitation, up to 21% of CD8+ lymphocytes secreted cytokines in response to antigen stimulation in immunized mice, as measured by ICS) at both the systemic and mucosal level when administered subcutaneously. Long-term payload expression by lentiviral vector-transduced cells resulted in sustained anti-HIV immunogenicity. Protocol combinations of the viral vectors with DNA plasmids in a DNA prime/lentiviral vector boost protocol resulted in greatly increased, polyfunctional anti-HIV cellular immunity, outperforming those elicited by Ad5 derived adenoviral vectors expressing similar payloads in side by side comparisons.

In general, the methods of the present invention preferably are employed to treat viral diseases that result from viral infection. Desirably, a virus (as well as the vector, as discussed below) is an RNA virus, but also can be a DNA virus. RNA viruses are a diverse group that infect prokaryotes (e.g., the bacteriophages) as well as many eukaryotes, including mammals and, particularly, humans. Most RNA viruses have single-stranded RNA as their genetic material, although at least one family has double-stranded RNA as the genetic material. The RNA viruses are divided into three main groups: the positive-stranded viruses (i.e., those of which the genome transferred by the virus is translated into protein, and whose deproteinized nucleic acid is sufficient to initiate infection), the negative-stranded viruses (i.e., those of which the genome transferred by the virus is complementary to the message sense, and must be transcribed by virion-associated enzymes before translation can occur), and the double-stranded RNA viruses. The method of the present invention preferably is employed to treat positive-stranded viruses, negative-stranded viruses, and double-stranded RNA viruses. The practice of the techniques described herein will employ, unless otherwise indicated, conventional techniques of cell biology, cell culture, molecular biology, genetics, microbiology, recombinant DNA, and immunology, which are within the skill of the art.

Pharmaceutical Compositions

The pharmaceutical compositions of the present invention contain a pharmaceutically and/or therapeutically effective amount of at least one nucleic acid construct, lentiviral vector, lentiviral vector system, viral particle/virus stock, or host cell (i.e., agents) of the invention. In one embodiment of the invention, the effective amount of an agent of the invention per unit dose is an amount sufficient to cause the detectable expression of the gene of interest. In another embodiment of the invention, the effective amount of agent per unit dose is an amount sufficient to prevent, treat or protect against deleterious effects (including severity, duration, or extent of symptoms) of the disease or condition being treated.

The administration of the pharmaceutical compositions of the invention may be for either “prophylactic” or “therapeutic” purpose. When provided prophylactically, the compositions are provided in advance of any symptom. The prophylactic administration of the composition serves to prevent or ameliorate any subsequent deleterious effects (including severity, duration, or extent of symptoms) of the disease or condition being treated. When provided therapeutically, the composition is provided at (or shortly after) the onset of a symptom of the condition being treated.

In yet another embodiment of the present invention, for all therapeutic, prophylactic and diagnostic uses, one or more of the aforementioned lentiviral vectors, lentiviral vector system, viral particle/virus stock, or host cell (i.e., agents) of the present invention, as well as other necessary reagents and appropriate devices and accessories, may be provided in kit form so as to be readily available and easily used. Such a kit would comprise a pharmaceutical composition for in vitro or in vivo administration comprising a lentiviral vector of the present invention, and a pharmaceutically acceptable carrier and/or a genetic adjuvant; and instructions for use of the kit.

Vaccine formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials. Extemporaneous injection solutions and suspensions may be prepared from purified nucleic acid preparations for the DNA plasmid priming compounds and/or purified viral vector compounds commonly used by one of ordinary skill in the art. Preferred unit dosage formulations are those containing a dose or unit, or an appropriate fraction thereof, of the administered ingredient. It should be understood that in addition to the ingredients, particularly mentioned above, the formulations may also include other agents commonly used by one of ordinary skill in the art.

The vaccine formulation may be administered through different routes, such as oral, including buccal and sublingual, rectal, parenteral, aerosol, intranasal, intramuscular, subcutaneous, intravenous, intraperitoneal, intraocular, intracranial, intradermal, transdermal (skin patches), topical, or direct injection into a joint or other area of the subject's body. The vaccine may likewise be administered in different forms, including but not limited to solutions, emulsions and suspensions, microspheres, particles, microparticles, nanoparticles, and liposomes. It is expected that from about one to about five dosages (e.g., two dosages—an initial inoculation, the prime of prime/boost, and a booster) may be required per immunization protocol. The initial prime and boost administrations may contain a quantity of antigen sufficient to induce a satisfactory immune response. An appropriate quantity of prime and boost antigen(s) to be administered is determined for any of the prime/boost protocols disclosed herein by one skilled in the art based on a variety of physical characteristics of the subject or patient, including, for example, the patient's age, body mass index (weight), gender, health, immunocompetence, and the like. Similarly, the volume of administration will vary depending on the route of administration. By way of example, intramuscular injections may range from about 0.1 mL to 1.0 mL. Preferably a patient has a normal immune system and is not infected with human immunodeficiency virus, although the vaccine may also be administered after initial HIV infection to ameliorate disease progression, or after initial infection to treat AIDS.

The vaccine may be stored at temperatures of from about −80° C. to about 37° C. or less, depending on how the vaccine is formulated. A variety of adjuvants known to one of ordinary skill in the art may be administered in conjunction with the viral vector in the vaccine composition.

One skilled in the art can easily determine the appropriate dose, schedule, and method of administration for the exact formulation of the composition being used, in order to achieve the desired “effective level” in the individual patient. One skilled in the art also can readily determine and use an appropriate indicator of the “effective level” of the compounds of the present invention by a direct (e.g., analytical chemical analysis) or indirect (e.g., with surrogate indicators of viral infection, such as p24 or reverse transcriptase in the case of HIV infection) analysis of appropriate patient samples (e.g., blood and/or tissues).

Further, with respect to determining the effective level in a patient for treatment of HIV, in particular, suitable animal models are available and have been widely implemented for evaluating the in vivo efficacy against HIV of various gene therapy protocols (Sarver et al. (1993b), supra). These models include mice, monkeys, cats and rabbits. Even though these animals are not naturally susceptible to HIV disease, chimeric mice models (e.g., SCID, bg/nu/xid, NOD/SCID, SCID-hu, immunocompetent SCID-hu, bone marrow-ablated BALB/c) reconstituted with human peripheral blood mononuclear cells (PBMCs), lymph nodes, fetal liver/thymus or other tissues can be infected with lentiviral vector or HIV, and employed as models for HIV pathogenesis and gene therapy. Similarly, the simian immune deficiency virus (SIV)/monkey model can be employed, as can the feline immune deficiency virus (FIV)/cat model. The pharmaceutical composition can contain other pharmaceuticals, in conjunction with a vector according to the invention, when used to therapeutically treat AIDS. These other pharmaceuticals can be used in their traditional fashion (i.e., as agents to treat HIV infection).

The vector of the present invention may be administered through various routes, including, but not limited to, oral, including buccal and sublingual, rectal parenteral, aerosol, nasal, intravenously, subcutaneous, intradermal and topical. In kinetic studies of the immune response generated by vaccine candidates, immune responses were maintained over time.

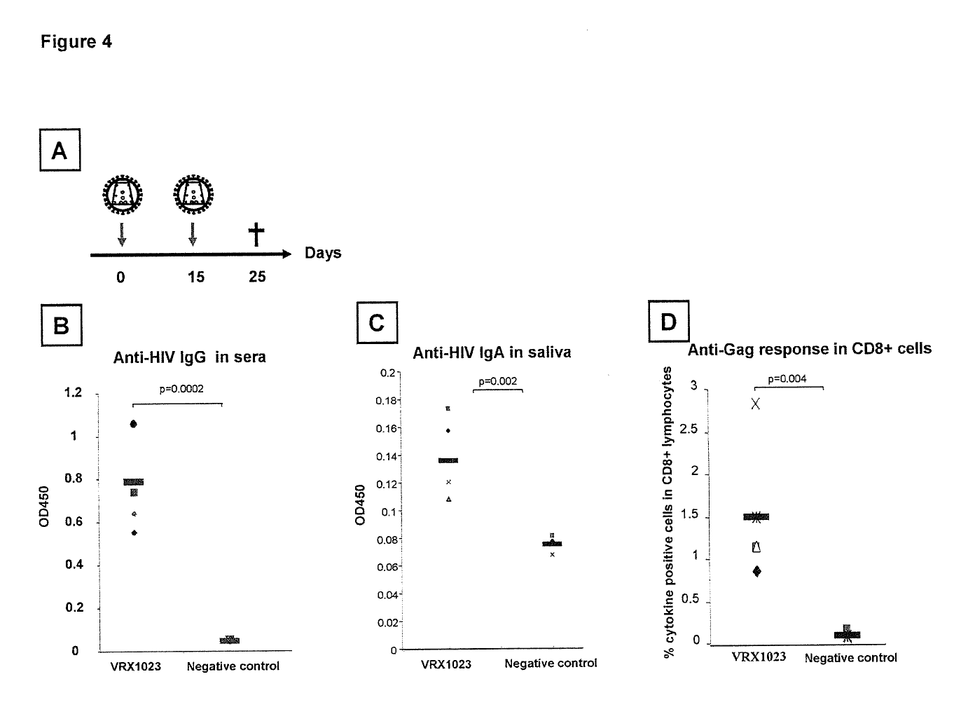

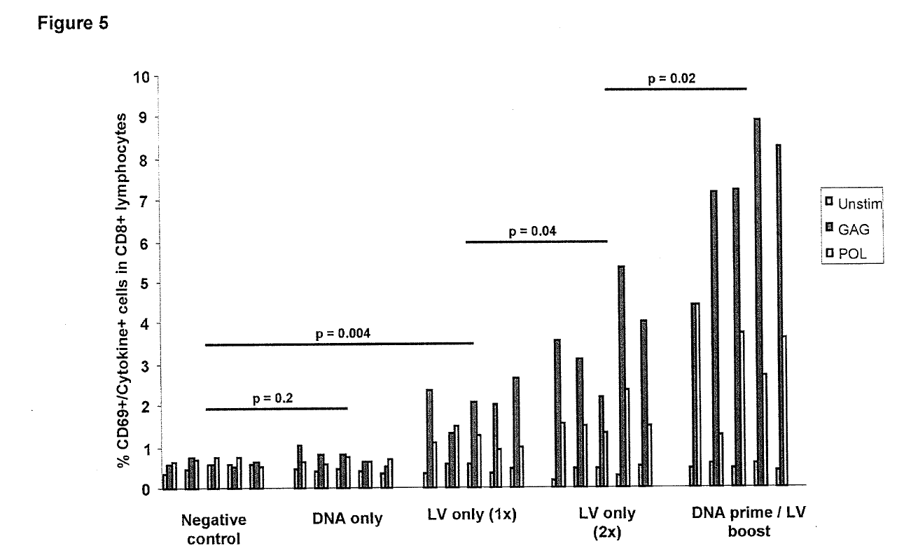

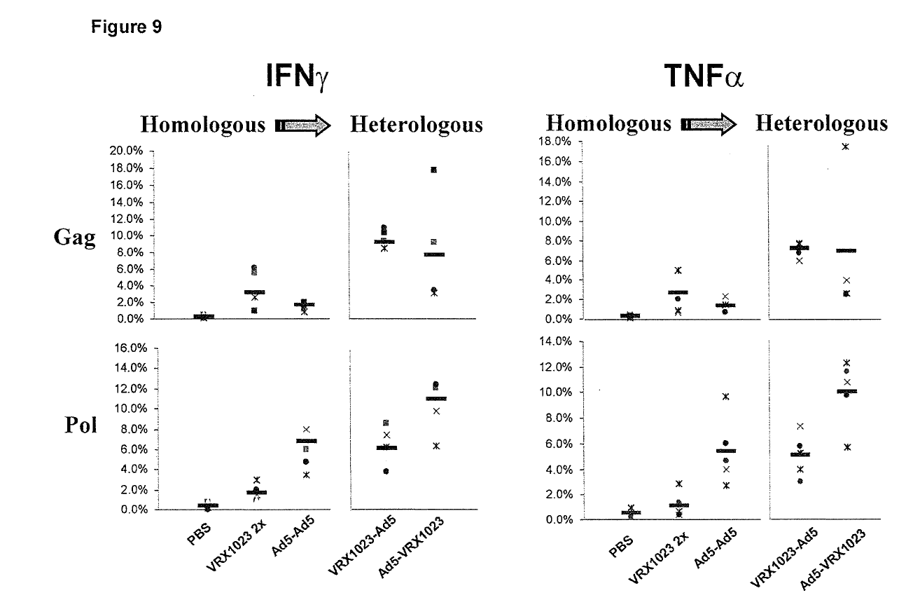

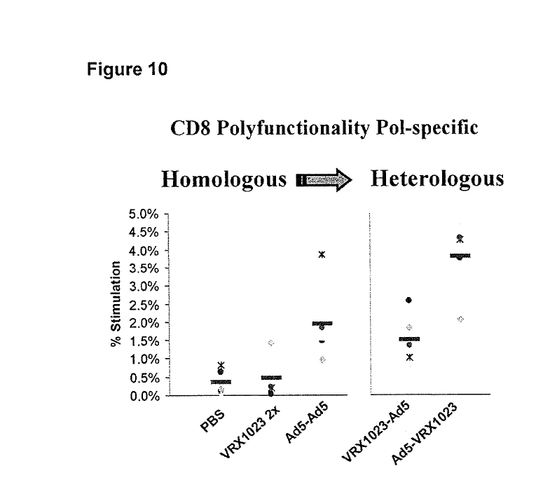

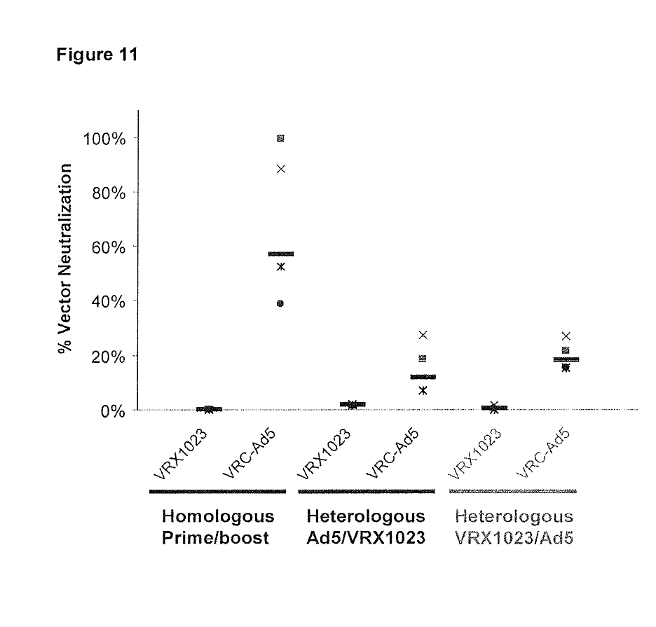

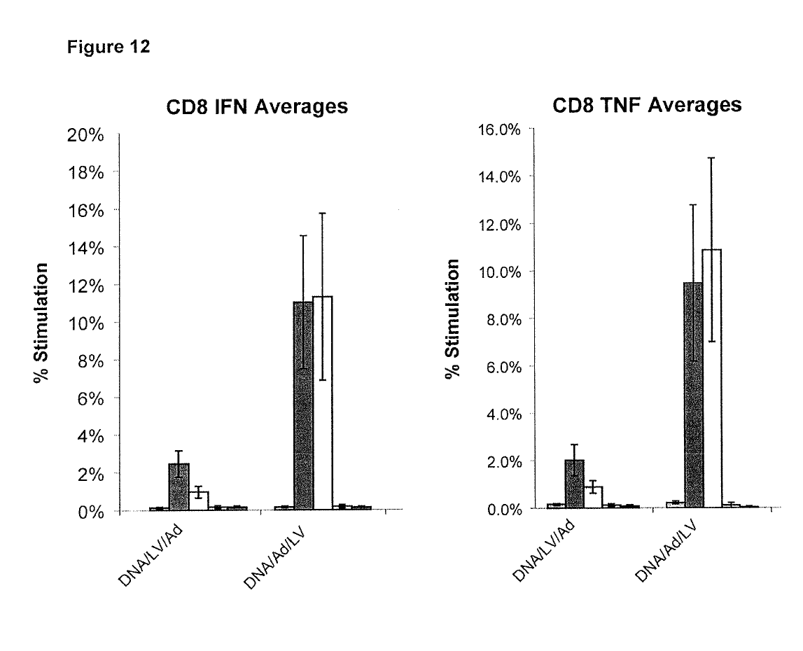

An effective administration route that elicited systemic and mucosal immunity at both the cellular and humoral levels was determined (FIG. 4). Later, the vaccine protocols were further modified to include either a DNA prime/lentiviral boost (FIG. 5), or consisting of vector prime/vector boost regimens, in which adenoviral and lentiviral vectors are used alternatively in homologous or heterologous prime/boost strategies to improve vaccine immunogenicity (FIGS. 9 and 10). These two protocols can alternatively be combined in a DNA prime, followed by two vector boosts (FIG. 12). All of these vaccination strategies can provide a significant advantage, as DNA vaccines are known to induce strong cellular immune responses, see, e.g., Nagata, Aoshi et al. 2004, and the emergence of an anti-vector immune response is obviated when an adenoviral boost is followed by a second boost comprising one of the lentiviral vectors disclosed herein (FIG. 11). Vaccine efficacy was studied using ICS and Elisa.

The invention now being generally described, it will be more readily understood by reference to the following examples, which are included merely for purposes of illustration of certain aspects and embodiments of the present invention, and are not intended to limit the invention. The contents of any patents, patent applications, patent publications, or scientific articles referenced anywhere in this application are herein incorporated in their entirety.

### EXAMPLES

### Example 1

In this example, it was demonstrated that the highly immunogenic lentiviral vectors of the present invention elicit long-term anti-HIV immunity in mice, particularly when used in a prime/boost protocol.

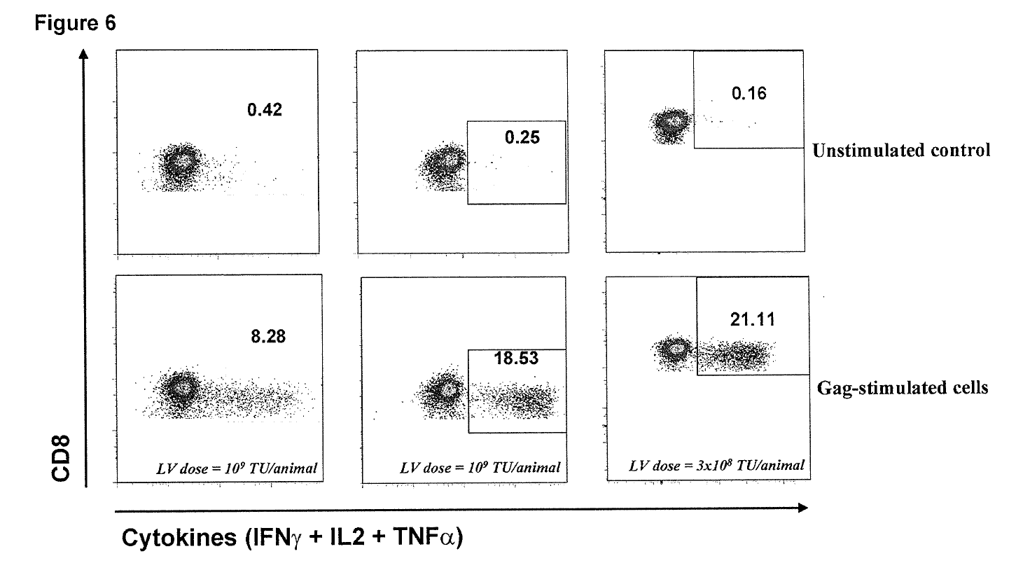

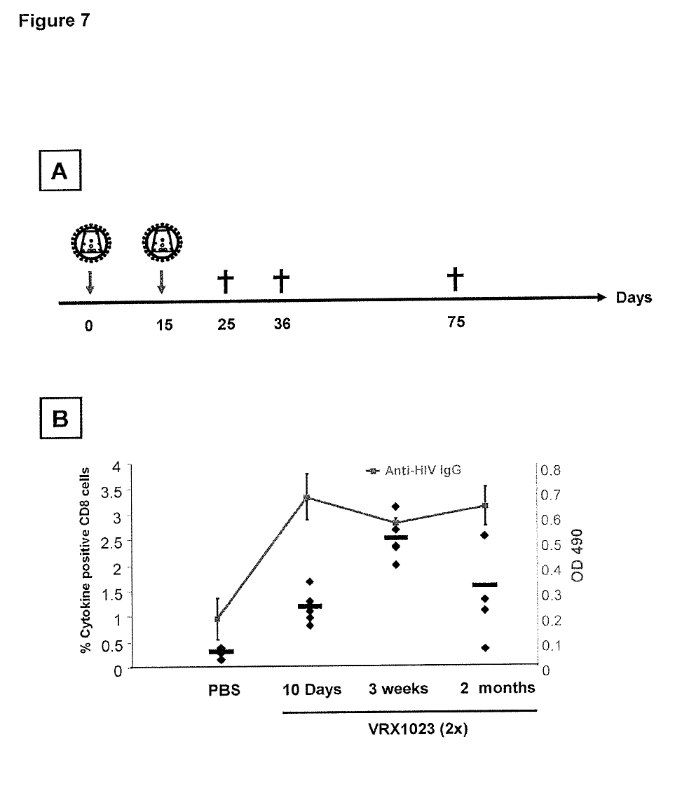

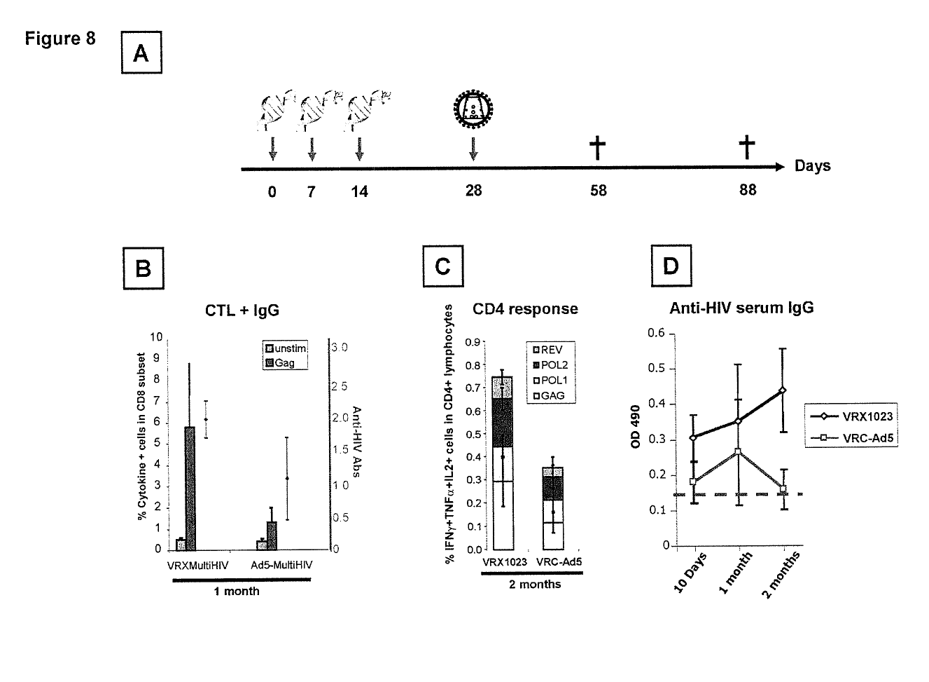

Among the current arsenal of genetic immunization tools, viral vectors, and especially lentiviral vectors, have demonstrated great promise. Engineered lentiviral vectors can infect a wide variety of cell types and, as opposed to other vectors, they can transduce even non-actively replicating cells, including dendritic cells, thus eliciting strong immunogenicity to the antigens which is beneficial to the treatment of a variety of diseases. Lentiviral vector constructs were modified to be used as vehicles to deliver HIV-derived antigens. A VSV-G pseudotyped, HIV-based lentiviral vector carrying the full-length Gag, Pol and Rev genes from HIV and driven by the native HIV 5′ LTR was used in the lentiviral vector of the present invention. A person of skill in the art would know how to modify the lentiviral vectors of the invention to create vaccines to any other diseases that are caused by an infections agent and/or cancer. Lentiviral vector development included route optimization in order to elicit both systemic and mucosal responses in mice, protocol optimization by combining the lentiviral vector and DNA plasmids in prime/boost protocols, and assessment of sustained immunogenicity. All studies were performed in mice and non-human primates and anti-HIV immunogenicity was assessed by ICS and Elisa. Extremely high magnitude responses relative to current HIV vaccines (for example, and not by way of limitation, up to 21% of CD8+ lymphocytes secreted cytokines in response to HIV antigen stimulation in immunized mice (FIG. 6)), from a variety of prime/boost protocols using the constructs of the invention. Subcutaneous immunization induced high-level anti-HIV humoral and cellular immunity in both the systemic and mucosal compartments (FIG. 4). Long-term antigen expression by lentiviral vector-transduced cells resulted in sustained anti-HIV immunogenicity (FIG. 7). Combinations of viral vectors with DNA plasmids in a DNA prime/lentiviral vector boost protocol resulted in greatly increased, polyfunctional anti-HIV cellular immunity, outperforming immune responses elicited by DNA prime/adenoviral vector boost protocols expressing similar payloads in side by side comparisons (FIG. 8).

### Example 2

In this example, it was demonstrated that immunization with HIV-based lentiviral vectors resulted in minimal anti-vector neutralization activity.

Viral (adenoviral and lentiviral) vectors represent one of the most attractive means of vaccination, as they typically induce strong antigen immunity. However, due to the adenoviral vector's widespread existence in nature it is not unusual for adenoviral vector based vaccines to generate immune responses to the adenoviral vector itself. These anti-adenoviral vector responses, either due to pre-existing immunity against the wild type adenovirus or induced by adenoviral vector administration, have been extensively described for a variety of adenoviral vectors, and can result in vaccine neutralization. This vaccine neutralization results in dramatic decrease of vaccine efficacy, and vaccine vectors overcoming this obstacle are needed.

It was assessed whether repeated administrations of the lentiviral vectors of the present invention induced vaccine neutralization, and whether anti-vector antibodies prevented subsequent immunizations. To test the neutralizing antibody responses in animal serum samples, a quantitative in vitro microtiter assay was developed by comparing levels of fluorescence of transduced Jurkat cells transduced with GFP-expressing lentiviral vectors pre-incubated with animal sera collected prior and after vaccination. Sera from mice and non-human primates collected longitudinally were analyzed after repeated lentiviral vector administrations using various routes. Even though repeated immunizations (both subcutaneous, intraperitoneal, or intravenous) increased anti-lentiviral vector neutralizing activities compared to single injections, anti-vector neutralizing titers remained minimal compared to those elicited by highly antigenic adenoviral based vaccine vectors (FIG. 11). Compared to Intra-peritoneal injections, subcutaneous administrations induced similar anti-lentiviral vector neutralizing antibodies while eliciting much stronger anti-payload immunity. Lentiviral vectors can be an alternative to other widely used viral vectors, when preexisting immunity or repeated administration is an issue. The lentiviral vectors of the present invention can be used alone or in various combinations with other vectors and DNA plasmids in a broad target population.

### Example 3

In this example, it was demonstrated that heterologous boosting in prime/boost protocols utilizing the lentiviral vectors of the invention enhanced the immunogenicity of an adenoviral vaccine vector and circumvented vaccine neutralization.

Heterologous “prime-boost” protocols with two different viral vectors have been shown to markedly increase the immunogenicity of the individual viral vectors, and minimize the generation of vaccine neutralizing activity. Pseudotyped lentiviral vectors are one of the most effective vehicles for delivering antigen payloads since they can infect a wide variety of cells, and possess the ability to transduce dividing as well as non-dividing cells. The lentiviral vector-based vaccine disclosed herein was tested for immunogenicity using a heterologous prime/boost strategy with an adenoviral based vector vaccine.

One of the lentiviral vectors of the present invention was engineered to carry full-length HIV Gag, Pol and Rev genes, and pseudotyped with VSV-G. Immunizations using this lentiviral vector and an adenoviral based vaccine vector were performed on mice, and immunogenicity assayed with ICS, as well as anti-HIV ELISA. Neutralization assays were carried out using the adenoviral based vaccine vector or the lentiviral vector to transduce Hela-tat cells in the presence of sera from immunized mice.

Combining an adenoviral vector-based vaccine vector and the lentiviral vaccine vector in a heterologous prime/boost approach induced a several-fold higher immunogenic response in CD8+ T-cells and greater polyfunctional response in CD4+ T cells, relative to homologous prime/boost immunizations (FIGS. 9 and 10). In addition, homologous prime/boost protocols were found to induce high levels of anti-vaccine neutralizing antibodies in adenoviral vector immunized mice, while an equally immunogenic lentiviral vector elicited much lower levels of neutralizing antibodies (FIG. 11). However, it was found that the heterologous prime-boost strategy did not elicit any significant neutralizing activity to lentiviral vector, while dramatically decreasing the anti-adenoviral vector neutralizing activity (FIG. 11). Furthermore, combining a lentiviral vaccine vector and an adenoviral vector-based vaccine vector in a heterologous boost protocol dramatically improves immunogenicity and polyfunctionality (i.e., the capability of T-cells to secrete multiple cytokines simultaneously in response to antigenic stimulation) of both vectors (FIGS. 9 and 10). These findings have major implications on the development of HIV vaccines and vaccines against other diseases as well. The vaccines and protocols of the present invention can be used to treat people with pre-existing immunity to other viral vectors such as adenoviral based vaccine vectors.

### Example 4

In this example, it was demonstrated that the lentiviral-based vaccine vectors of the invention elicit strong and sustained anti-HIV cellular and humoral immunity in mice and non-human primates.

Viral vectors are considered as one of the major means for the induction of strong immune responses against recombinant antigens by genetic immunization. Among these, lentiviral vectors were engineered in order to infect a wide variety of cell types and, as opposed to other vectors, they can transduce even non-actively replicating cells, including dendritic cells, one of the major players for immune response induction.

As more fully described herein, lentiviral vectors of the present invention can induce a diversified type of immunity as is shown in the schematic diagrams of FIGS. 4B, 4C and 4D. FIG. 4B shows anti-HIV IgG levels induced by a lentiviral vector of the present invention as compared to an “empty” lentiviral vector control. FIG. 4B shows significant sera IgG levels post subcutaneous injection of the vector of the present invention in Balb/c mice. The results obtained and illustrated in FIG. 4C showed significant anti-HIV IgA levels in mouse saliva while FIG. 4D shows that the lentiviral vector induces significant anti-HIV responses in CD8+ T cells. The results obtained are significant for the therapeutic and prophylactic treatment of disease, particularly HIV, in that a diverse immunity is observed systemically to therapeutically treat progression and mucosally to prophylactically treat the area of transmission as in the case of HIV or other viruses for which the invention can be adapted. This is key to the therapeutic and prophylactic treatment of HIV. The data showed that the subcutaneous route was the most effective at inducing both systemic and mucosal immunity to HIV.

Although it is a feature of the invention that it can be used alone as a prophylactic and therapeutic vaccine, the vaccine protocols were modified to include a DNA prime-lentiviral boost strategy to improve vaccine immunogenicity as well as a lentiviral prime-lentiviral boost strategy. This presents a significant advantage, as DNA vaccines induce strong and focused cellular immune responses, see, e.g., Nagata, Aoshi et al. 2004. The DNA prime-lentiviral vector strategy was shown to be an effective means of eliciting a potent systemic immune response; particularly for generating cell-mediated immunity against Gag, a factor that has most been correlated with HIV+ non-progression. See, e.g., Barouch, Fu et al. 2001; Letvin, Barouch et al. 2002; Barouch, McKay et al. 2003; Betts, Nason et al. 2006; and Honeyborne, Prendergast et al. 2007. In addition, lentiviral vectors used alone as well as in DNA prime-lentiviral vector boost approaches, counter the most significant problem encountered with other viral vectors; the generation of anti-vector immunity post-immunization.

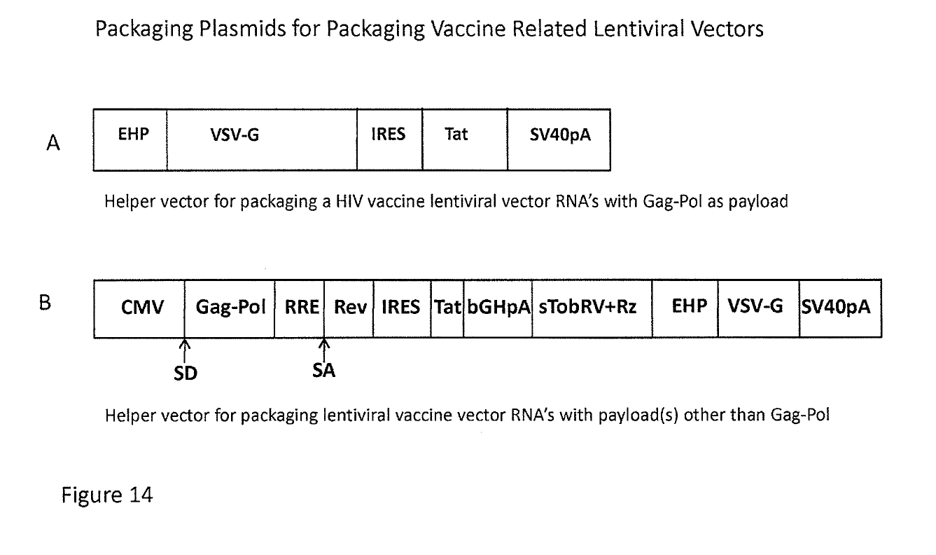

The present invention provides for successful combination of the lentiviral vector vaccination with a DNA in a DNA prime/lentiviral vector boost setting. The effects of a DNA prime/lentiviral boost were studied. FIG. 5 discloses that DNA prime/lentiviral vector boost increases HIV-specific CD8+ response. A naked DNA plasmid (e.g. FIG. 14) having the same payload as the lentiviral vector (e.g. FIG. 1) was made. An ICS assay of mice splenocytes to measure cell-mediated (CD8+) response was performed. The results showed that boost strategies, either DNA prime or lentiviral vector prime (homologous prime/boost) using the lentiviral vector of the invention as the boost showed increased immunogenicity. In each instance, the prime and boost were constructed to carry the same payload.

It is an aspect of the invention to provide for a high magnitude of anti-HIV immunity. Using an ICS assay to measure immune response, FIG. 6 shows that a significant magnitude of stimulation of anti-HIV immunity can be elicited by lentiviral vector vaccination after a DNA prime. The lentiviral vector of the present invention elicited an anti-HIV Gag-specific response in 21% of CD8+ T lymphocytes. These results were confirmed repetitively in various experiments via flow cytometry.

Lentiviral vector immunization induces sustained, long-term anti-HIV immunity. One of the challenges of addressing HIV infection is the ability to induce sustained anti-HIV immunity on the long term. FIG. 7 shows anti-HIV Gag responses elicited by the lentiviral vector of the present invention in CD8+ cells and anti-HIV IgG responses in mouse sera. Results demonstrate that the lentiviral vector of the present invention induces sustained immunogenicity throughout the study. Additional testings with up to 9 months follow-up show that immunity is sustained for the whole duration of the study. Sustained antigen expression by LV is also demonstrated in FIG. 8D, where LV-induced antibody responses increase over time, while those elicited by adenoviral vectors decline.

FIG. 8 shows comparisons of the lentiviral vector vaccine of the present invention with other adenovirus-based vaccine candidates. The results exemplified in FIG. 8 showed that, the lentiviral vaccine of the present invention induced a higher anti-HIV cellular and humoral immunity than the one elicited by the adenovirus vaccine. FIG. 8C shows the polyfunctionality of HIV-specific CD4 cells elicited by the present invention. The results showed that for each HIV antigen CD4+ cells showed a marked increase over an adenovirus vaccine in CD4 immune response. Moreover, the results of FIG. 8D show sustained and increasing anti-HIV serum antibodies, compared to the adenoviral platform.

FIG. 11 illustrates anti-vector neutralizing activity generated by viral vaccine vector homologous and heterologous prime/boost protocols. The Y axis represents the percentage of viral vector that is neutralized by the host's antibodies. Results demonstrate that while the lentiviral vaccine of the present invention induces similar or higher anti-HIV immunity than its adenoviral counterpart (FIGS. 8 and 9), it is much less susceptible to anti-vector immunity, and therefore its combination to adenoviral vaccine in heterologous prime/boost can bypass the anti-adenovirus neutralization issue.

FIG. 12 illustrates CD8+ immune response to two HIV antigens, Gag and Pol, by measuring the percent of CD8+ cells that secrete cytokines, (INFγ or TNFα), in response to stimulation with the Gag and Pol antigens. These CD8+ cells came from mice that were vaccinated with two different DNA prime/heterologous boost protocols. All immunogens used, DNA and viral vectors, contained the same payload.

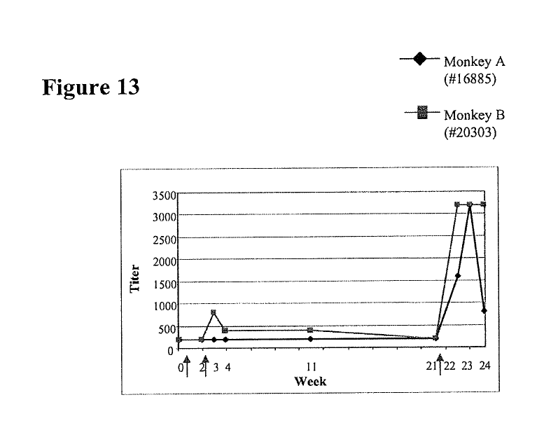

FIG. 13 illustrates anti-SIV antibody titers generated in non-human primates after an SIV (primate equivalent of HIV)-based lentiviral vaccine vector was administered three times over five months. The boosting effect observed after the third administration reveals a minimal anti-vector response, even after multiple administrations. This illustrates that the lentiviral based vaccine vectors of the invention do not elicit anti-vector neutralizing antibodies that would render the vaccine ineffective after numerous administrations, as this is a major obstacle to the use of other viral vector vaccines.

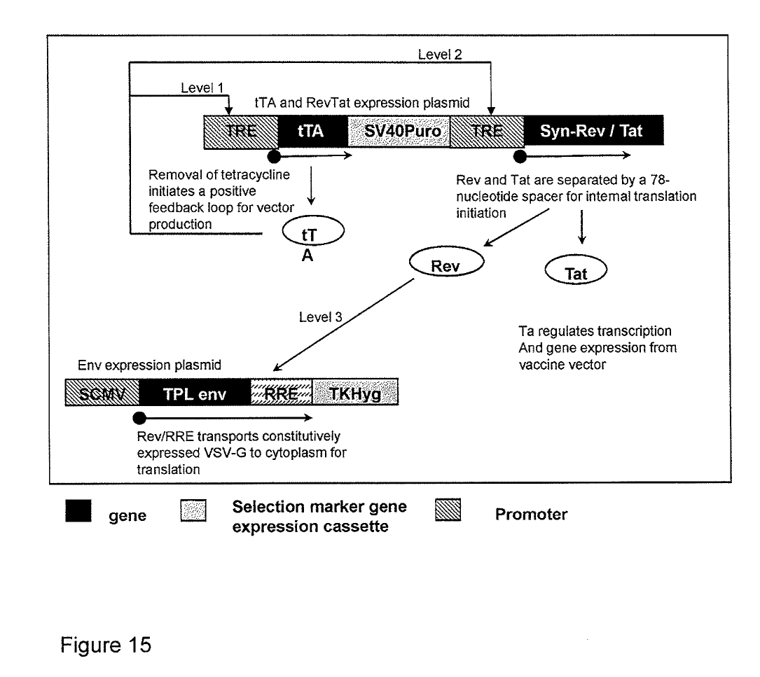

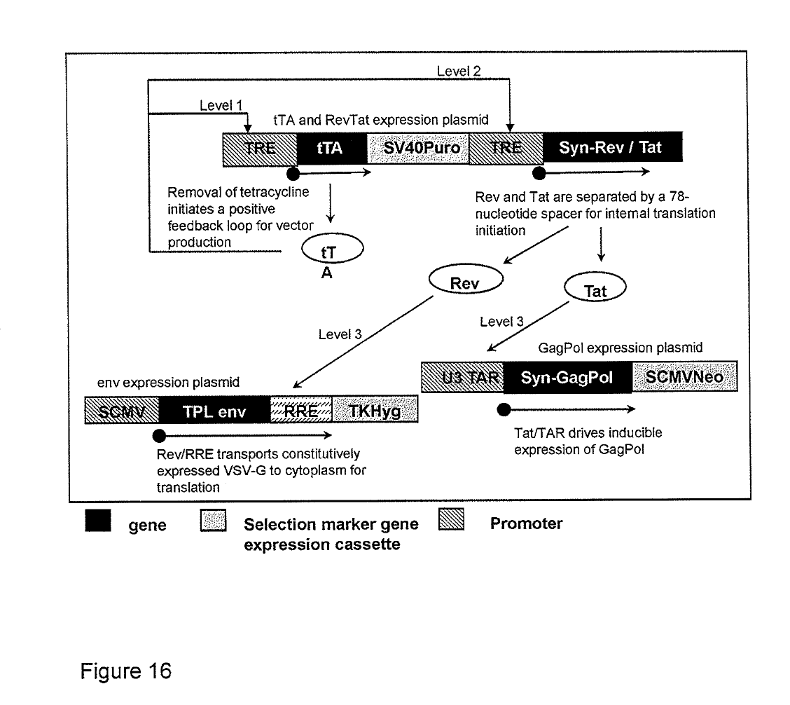

FIGS. 15 and 16 illustrate packaging cell line helper constructs for vaccine lentiviral vector RNA's packaging where gag/pol is the payload in the vaccine vector (FIG. 15) and where gag/pol are not the payload in the vaccine vector (FIG. 16).

One skilled in the art will appreciate that numerous equivalents of the foregoing materials and equipment are readily available and that these Examples may be modified in accordance with the principles hereof using no more than routine experimentation. All references cited herein, including patents, patent applications, and publications, are hereby incorporated by reference in their entireties, whether previously specifically incorporated or not.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

### REFERENCES

Barouch, D. H., A. Craiu, et al., “Elicitation of high-frequency cytotoxic T-lymphocyte responses against both dominant and subdominant simian-human immunodeficiency virus epitopes by DNA vaccination of rhesus monkeys,” J. Virol. 75(5), 2462-67 (2001).
Barouch, D. H., T. M. Fu, et al., “Vaccine-elicited immune responses prevent clinical AIDS in SHIV(89.6P)-infected rhesus monkeys,” Immunol. Lett. 79(1-2), 57-61 (2001).
Barouch, D. H. and N. L. Letvin, “CD8+ cytotoxic T lymphocyte responses to lentiviruses and herpesviruses,” Curr. Opin. Immunol. 13(4), 479-82 (2001).
Barouch, D. H., P. F. McKay, et al., “Plasmid chemokines and colony-stimulating factors enhance the immunogenicity of DNA priming-viral vector boosting human immunodeficiency virus type 1 vaccines,” J. Virol. 77(16), 8729-35 (2003).
Betts, M. R., M. C. Nason, et al., “HIV nonprogressors preferentially maintain highly functional HIV-specific CD8+ T cells,” Blood 107(12), 4781-89 (2006).
Blazevic, V., A. Mannik, et al., “Induction of human immunodeficiency virus type-1-specific immunity with a novel gene transport unit (GTU)-MultiI IIV DNA vaccine,” AIDS Res. Hum. Retroviruses 22(7), 667-77 (2006).
Brander, C., N. Frahm, et al., “The challenges of host and viral diversity in HIV vaccine design,” Curr. Opin. Immunol. 18(4), 430-37 (2006).
Butler, I. F., I. Pandrea, et al., “HIV genetic diversity: biological and public health consequences,” Curr. HIV Res. 5(1), 23-45 (2007).
Ferrari, G., W. Neal, et al., “Absence of immunodominant anti-Gag p17 (SL9) responses among Gag CTL-positive, HIV-uninfected vaccine recipients expressing the HLA-A*0201 allele,” J. Immunol. 173(3), 2126-33 (2004).
Holodniy, M., “HIV-1 load quantitation: a 17-year perspective,” J. Infect. Dis. 194 Suppl 1: S38-44 (2006).
Honeyborne, I., A. Prendergast, et al., “Control of human immunodeficiency virus type 1 is associated with HLA-B*13 and targeting of multiple gag-specific CD8+ T-cell epitopes,” J. Virol. 81(7), 3667-72 (2007).
Komanduri, K. V., S. M. Donahoe, et al., “Direct measurement of CD4+ and CD8+ T-cell responses to CMV in HIV-1-infected subjects,” Virology 279(2), 459-70 (2001).
Letvin, N. L., D. H. Barouch, et al., “Prospects for vaccine protection against HIV-1 infection and AIDS.” Annu. Rev. Immunol. 20, 73-99 (2002).
Letvin, N. L., J. R. Mascola, et al., “Preserved CD4+ central memory T cells and survival in vaccinated SIV-challenged monkeys.” Science 312(5779), 1530-33 (2006).
Levine, B. L., L. M. Humeau, et al., “Gene transfer in humans using a conditionally replicating lentiviral vector.” Proc. Nat'l Acad. Sci. (U.S.A.) 103(46), 17372-77 (2006).
Marshall, E., “Clinical research. Gene therapy a suspect in leukemia-like disease.” Science 298(5591), 34-35 (2002).
McBurney, S. P. and T. M. Ross, “Developing broadly reactive HIV-1/AIDS vaccines: a review of polyvalent and centralized HIV-1 vaccines,” Curr. Pharm. Des. 13(19), 1957-64 (2007).
Nagata, T., T. Aoshi, et al., “Cytotoxic T-lymphocyte-, and helper T-lymphocyte-oriented DNA vaccination.” DNA Cell Biol. 23(2), 93-106 (2004).

What is claimed is:

1. A lentiviral vector comprising
a 5′ long terminal repeat (LTR) and a 3′ LTR;
a first nucleic acid sequence operably linked to said 5′ LTR;
a second nucleic acid sequence operably linked to said 5′ LTR comprising a functional REV coding sequence and a rev response element (RRE)-containing sequence wherein the RRE-containing sequence is located upstream of the REV coding sequence; and
wherein transcription of said first nucleic acid sequence and said second nucleic acid sequence is driven by said 5′ LTR.

2. The vector of claim 1, further comprising a nucleic acid sequence encoding one or more functionally active lentiviral RNA packaging elements.

3. The vector of claim 1, further comprising a nucleic acid sequence encoding functional central polypurine tract (cPPT), and central termination sequence (cTS), and 3′ LTR proximal polypurine tract (PPT) elements.

4. The vector of claim 1, wherein said first nucleic acid sequence encodes one or more antigenic sequences of interest.

5. The vector of claim 4, wherein expression of said one or more antigenic sequences of interest depends on REV-RRE activity.

6. The vector of claim 1, wherein said first nucleic acid sequence comprises a Gag/Pol coding sequence or derivative thereof.

7. The vector of claim 6, wherein said first nucleic sequence is an unmodified sequence.

8. The vector of claim 6, wherein said Gag/Pol coding sequence comprises a modified Gag/Pol coding sequence.

9. The vector of claim 6, wherein said cPPT/cTS is a part of a Pol coding sequence of said Gag/Pol coding sequence.

10. The vector of claim 2, wherein said functionally active lentiviral RNA packaging elements comprise a Gag packaging sequence or derivative thereof.

11. The vector of claim 5, further comprising a heterologous promoter located 3′ of said RRE, wherein said heterologous promoter comprises a viral promoter, a human promoter, or a synthetic promoter or a combination thereof.

12. A pharmaceutical composition comprising a lentiviral vector according to claim 1.

13. A method for inducing an immune response in a subject comprising administering a pharmaceutical composition according to claim 12.

14. The method according to claim 13, wherein said lentiviral vector expresses one or more genes of interest to potentiate immunity, and wherein said immune response comprises a humoral immune response, a cell mediated immune response or a combination thereof.

15. The method according to claim 14 wherein said humoral immune response, cell mediated immune response or a combination thereof is specific to a disease or condition of interest comprising cancer, Alzheimer's disease, autoimmune diseases, cardiovascular diseases, neurological diseases, fibrotic diseases, lipid metabolism diseases, extra-cellular matrix-related diseases, and chronic joint degenerative diseases, or any combination thereof.

16. A method of increasing the immunogenicity of a vector in a host cell comprising administering a first vector, followed by one or more sequential administrations of a lentiviral vector according to claim 1.

17. A method of inhibiting or controlling the replication of an infective replicative human immunodeficiency virus (HIV) in a mammal in need thereof comprising administering a pharmaceutical composition comprising a lentiviral vector according to claim 1.

18. A method of increasing immunogenicity of a vector in a subject in need thereof comprising
a) administering a prime; and
b) sequentially administering a boost,
wherein at least one of said prime or said boost comprises a lentiviral vector according to claim 1.

19. A recombinant lentiviral packaging cell comprising
a first nucleic acid molecule capable of expressing, in said packaging cell, a nucleic acid sequence of interest to produce transduction-competent virus-like particles; and
wherein said cell produces no transduction-competent virus-like particles in the absence of a second nucleic acid molecule.

20. A method of producing a recombinant lentiviral packaging cell comprising
introducing into a cell, a nucleic acid capable of expressing in said packaging cell, a nucleic acid sequence to produce transduction-competent virus-like particles; and
at least one nucleic acid molecule capable of expressing the sequence of interest in said packaging cell, wherein said packaging cell produces transduction-competent virus-like particles expressing the nucleic acid sequence of interest.
